(19) United States
(12) Patent Application Publication
(10) Pub. No.: US 2026/0216229 A1
(43) Pub. Date: Jul. 30, 2026

(54) PROGRAMMABLE DNA PROTEOLYSIS TARGET CHIMERAS AND METHODS OF THEIR USE

(71) Applicant: ARIZONA BOARD OF REGENTS ON BEHALF OF ARIZONA STATE UNIVERSITY, Scottsdale, AZ (US)

(72) Inventors: Hao YAN, Gilbert, AZ (US); Yang XU, Chandler, AZ (US); Rong ZHENG, Tempe, AZ (US); Abhay PRASAD, Tempe, AZ (US); Deeksha SATYABOLA, Tempe, AZ (US)

(21) Appl. No.: 19/151,888

(22) PCT Filed: Jan. 29, 2024
(86) PCT No.: PCT/US2024/013415

§ 371 Date: Jul. 29, 2025

Related U.S. Application Data
(60) Provisional application No. 63/441,956, filed on Jan. 30, 2023.

(51) Int. Cl. A61K 31/713; A61K 47/54; A61K 47/55; C07H 21/04

(52) U.S. Cl. CPC A61K 31/713; A61K 47/545; A61K 47/55; A61K 47/554; C07H 21/04

(57) ABSTRACT

Described herein are programmable DNA proteolysis target chimera complexes which can be used to both directly treat a cancer by inhibiting a biochemical pathway overexpressed in a cancer cell and indirectly treat a cancer by recruiting an E3 ligase complex to engage with a protein of interest or mutant thereof and commence proteolysis. Also described herein are methods of using said complexes for treating cancer, and compositions comprising said complexes.

### CROSS-REFERENCE TO RELATED APPLICATIONS

This application is a U.S. national phase of International Patent Application No. PCT/US2024/013425, filed Aug. 8, 2025, which claims priority to U.S. Provisional Application No. 63/441,956, filed Jan. 30, 2023, each of which are herein incorporated by reference in its entirety.

### FEDERALLY SPONSORED RESEARCH

This invention was made with government support under R01 GM155563 awarded by the National Institutes of Health. The government has certain rights in the invention.

### SEQUENCE STATEMENT

The instant application contains a Sequence Listing, which has been submitted electronically and is hereby incorporated by reference in its entirety. Said file, created on Jan. 22, 2024, is named G8118-03701_SL.xml and is 112,824 bytes in size.

### FIELD

This disclosure relates to compositions comprising Programmable DNA Proteolysis Target Chimeras (DNA-PROTAC) and methods of their use. Also described herein are methods of killing overly proliferative cells using said DNA-PROTACs, and DNA-PROTAC compositions.

### BACKGROUND

The human proteome comprises approximately 20,000 proteins, and it is estimated that more than 600 of them are functionally important for various types of cancer, including nearly 400 non-enzyme proteins that are challenging to target by traditional occupancy-driven pharmacology. Occupancy-driven pharmacology with small molecule inhibitors hinges on the prerequisite of target proteins having compatible binding pockets, rendering roughly 85% of the proteome “undruggable” and amplifying the risk of cumulative toxicities and drug resistance due to the requirement for high working concentrations.

The process of protein breakdown is crucial for preserving cellular homeostasis and is closely regulated. Through the ubiquitin-proteasome pathway (UPP), damaged, misfolded, or excess proteins can be identified and removed selectively. Actually, practically every cellular process is regulated by the UPP. Since the ubiquitin system controls essential elements of cell function, mutations in the process are the cause of a wide range of human illnesses, including cancer. The three phases of ubiquitination are as follows: (i) adding a ubiquitin polypeptide moiety to activate an enzyme called ubiquitin activating enzyme (E1); (ii) transferring a ubiquitin moiety to a cysteine residue on an enzyme called ubiquitin conjugating enzyme (E2); and (iii) formation of an isopeptide bond between the ubiquitin moiety and a lysine in the target protein, catalyzed by a ubiquitin ligase (E3). The specificity of the process is controlled by the E3 enzyme, which recognizes and interacts with the target protein to be degraded.

Small-molecule PROTACs (Proteolysis Targeting Chimeras) represent a new paradigm in pharmacology with the potential to be as transformative for cancer treatment as targeted kinase inhibitors, therapeutic antibodies, or immunotherapies (Békés, M.; Langley, D. R.; Crews, C. M. PROTAC Targeted Protein Degraders: The Past Is Prologue. Nature Reviews Drug Discovery. Nature Research Mar. 1, 2022, pp 181-200). A typical small-molecule PROTAC consists of a target protein-binding ligand, a E3 ligase recruiting ligand and a chemical linker such as polyethylene glycol (PEG) or alkyl chain that connects both the ligands. The small molecule-PROTAC mediated recruitment of the E3 ligase to the targeted protein induces ubiquitination and subsequent protein degradation via the proteasome. Different from the conventional small molecule-based drugs used in clinics that directly inhibit the target proteins, PROTACs simultaneously block the enzymatic and non-enzymatic functions of the target proteins and induce degradation of the whole protein. Following the promising clinical trials results from first two PROTACs against cancer, several PROTAC degraders have been developed and have entered clinical trials.

PROTACs could dramatically change the field of drug discovery and lead to specific “event-driven” pharmacology. However, considerable challenges associated with traditional small-molecule PROTACs impede its uses in the clinical translation. Low solubility, poor permeability, and off-target toxicity limit the applicability of current chemical linker based PROTACs.

Recently, nucleic acid nanotechnology has emerged as a promising approach for cancer targeting and treatment.

### SUMMARY

As described in this disclosure, the present invention provides for a programmable DNA proteolysis target chimera complex comprising: (a) a first DNA strand containing one or a plurality of independent E3 ligase ligands; and (b) a second DNA strand comprising one or a plurality of independent protein of interest (POI) targeting ligands. At least a portion of the first DNA strand is complementary to a portion of the second DNA strand and the first and second DNA strands form a DNA duplex. The one or a plurality of independent E3 ligase ligands is connected to the first DNA strand at a selected position on said first DNA strand. The one or a plurality of independent POI targeting ligands is connected to the second DNA strand at a selected position on said second DNA strand.

In some aspects, the programmable DNA proteolysis target chimera complex further comprises a targeting moiety selected from a cell-penetrating peptide or a blood-brain barrier traversal agent. In some aspects, the blood-brain barrier traversal agent is a lipid or cholesterol derivative.

In some aspects, the plurality of independent POI targeting ligands target separate proteins. In some aspects, the plurality of independent POI targeting ligands target separate portions of the same protein. In some aspects, there are at least two independent POI targeting ligands. In some aspects, there are at least two independent E3 ligase ligands.

In some aspects, the E3 ligase ligand is covalently connected to the first DNA strand. In some aspects, an E3 ligase protein is complexed to the one or plurality of E3 ligase ligands

In some aspects, the POI targeting ligand is covalently connected to the second DNA strand. In some aspects, a protein of interest is complexed to the one or plurality of POI targeting ligands.

In some aspects, the selected position on said first DNA strand and the selected position on the second DNA strand are separated by a distance from about 0.99 nm to about 7 nm. In some aspects, the selected position on said first DNA strand and the selected position on the second DNA strand are separated by a rotational angle about the double-stranded DNA complex from about 36 to about 180 degrees. In some aspects, the selected position on said first DNA strand and the selected position on the second DNA strand are separated by a distance of about a minor groove to about a major groove.

In some aspects, the first DNA strand and second DNA strand independently comprise a nuclease resistance feature. In some aspects, the nuclease resistance feature is selected from a sugar modification or an internucleoside linkage modification. In some aspects, the sugar modification is selected from a locked nucleic acid, a threose nucleic acid, or a 2′-alkoxy modification. In some aspects, the internucleoside linkage modification is a phosphorothioate, phosphoroselenoate, or phosphoramidate.

In some aspects, the protein of interest is selected from: CDK6, CDK4, BCR-Abl, EGFR, BTK, BRD4, HDAC6, STAT3, BCL-XI, FAK, P38-alpha, myc, Arora, Ras, and Jak.

In some aspects, this disclosure provides for a method of killing a cancer cell, the method comprising contacting a programmable DNA Proteolysis target chimera complex as described herein with a cancer cell. In some aspects, the cancer cell is a glioblastoma cancer cell.

In some aspects, this disclosure provides for a method of treating cancer in a subject, the method comprising administering to the subject an effective amount of a programmable DNA Proteolysis target chimera complex as described herein.

In some aspects, this disclosure provides for a method of treating a proliferative disease or disorder in a subject, comprising administering to the subject in need thereof a therapeutically effective amount of a complex as described herein. In some aspects, the proliferative disease or disorder is cancer.

In some aspects, this disclosure provides for a method of reducing the proliferation of a cancer tumor cell, the method comprising contacting said cancer tumor cell with a complex as described herein.

In some aspects, this disclosure provides for the use of a complex as described herein for the manufacture of a medicament for the treatment of cancer in a subject.

In some aspects, this disclosure provides for a composition comprising a complex as described herein, and a pharmaceutically acceptable carrier.

In some aspects, this disclosure provides for the use of a composition comprising a complex as described herein for the manufacture of a medicament for treating a proliferative disease or disorder in a subject. In some aspects, the disease or disorder is cancer.

In some aspects, this disclosure provides for a composition comprising a complex as described herein for the prophylactic or therapeutic treatment of a disease or disorder in a subject. In some aspects, the disease or disorder is cancer.

In some aspects, the DNA-PROTAC comprises at least two DNA strands which are independently of about 20 nucleotides in length. In some aspects, the DNA-PROTAC comprises a DNA strand having at least about 75, 80, 85, 90, 95, 97, or 99%, or 100% sequence identity to SEQ ID NOs: 1-26. Sequences are described in the included Sequence ID listing provided with this disclosure. In some aspects, the DNA strand is further modified with a functional linker group. In some aspects, the functional linker group comprises an alkynyl moiety, an azide moiety, or a dibenzo-cyclooctyne (DBCO) moiety.

### BRIEF DESCRIPTION OF DRAWINGS

FIG. 1A shows the design of DNA-PROTAC conjugates. A ssDNA strand is functionalized with E3 ligase binding ligand and another complementary ssDNA with special positioning (different position is shown with blue ball) of POI (protein of interest) target binding ligand. After successful validation of DNA-PROTACs in the in vitro system, it can be functionalized with cell/organ-targeting small molecules (e.g., lipid molecule to cross the blood-brain barrier) to avoid off-target toxicity. In some embodiments, DNA duplexes are chemically modified with phosphorothioate nucleic acids at the terminal position to impede in vivo degradation.

FIG. 1B shows the design of a DNA-PROTAC for simultaneous multitargeting protein degradation.

FIG. 1C shows the design of a spatially programmable DNA-PROTAC demonstrating spatial programmability of the complex. The spatially controllable distance “x” and angle of the two ligands—E3-inhibitor (E3-i) and protein of interest (POI-i) along the DNA duplex can be modulated.

FIG. 1D shows the table of the distances and angles obtained from representative embodiments of spatially programmable DNA-PROTACs of this disclosure.

FIG. 1E depicts one embodiment of a multi-E3 recruiting DNA-PROTAC conjugate (multi-EDPC) utilizing aDNA Holliday junction junction. The DNA nanostructure can be modified with two E3-ligase binding ligands and one target ligand.

FIG. 1F shows one embodiment of a multi-target protein recruiting DNA-PROTAC conjugate (multi-TPDPC) utilizing a DNA Holliday junction junction. The DNA nanostructure can be modified with two different target ligands and one E3 ligase binding ligand.

FIG. 2A shows screened spatially programmable DTAC library. DNA-PROTAC designs prepared with increment in distance between E3 ligase and Protein of interest from Version 01 to Version 05.

FIG. 2B shows a 8% Native PAGE gel electrophoresis of the Version 01 to 05 demonstrated formation of a well-formed DNA-PROTAC library.

FIG. 3 shows Western blot analysis of CDK6 degradation in U251 cells treated with distance-based DNA-PROTACs. The upper panels show the levels of CDK6 protein after treatment with various concentrations of series DNA-PROTACs, and the lower panels display β-Tubulin as a loading control.

FIG. 4A shows Dose- and Time-dependent Degradation of CDK6 and CDK4 in U251 Cells. CDK6 and CDK4 protein levels under different doses of DNA-PROTAC-V02.

FIG. 4B shows CDK6 and CDK4 protein levels in cells treated with 50 nM DNA-PROTAC-V02 for specified time durations

FIG. 5 shows qualitative confocal analysis of CDK6 protein degradation in U251 cell where CDK6 degradation occurred most in DNA-PROTACs over control sequences of DNA-E3i and DNA-CDK6i and scrambled dsDNA, clearly demonstrating a synergistic effect of the CDK6 degradation of the DNA-PROTACs of this disclosure.

FIG. 6 shows proteasome dependent CDK6 degradation in U251 cells. The proteasome inhibitor, MG-132 was pre-incubated with cells prior to DNA-PROTAC-V02 transfection and assessed CDK6 levels after 16 h.

FIG. 7A shows DNA-PROTAC-V02-Mediated Degradation of CDK6 at the Protein Level. Western blot analysis illustrating the impact of DNA-PROTAC-V02 and control treatments on CDK6 expression.

FIG. 7B shows the CDK6 and CDK4 mRNA levels under DNA-PROTAC-V02 treatment.

FIG. 7C is a Western blot analysis demonstrating the effect of BSJ-03-123 treatment on CDK6 expression

FIG. 7D shows CDK6 and CDK4 mRNA levels under BSJ-03-123 treatment.

FIG. 8 shows Western blot analysis of different angular-based DNA-PROTACs. Western blot analysis illustrating the impact of variable angular-based DNA-PROTACs and control treatments on CDK6 expression.

FIGS. 9A and 9B show H1 and C13 NMR spectra for selected E3 ligand, Pomalidomide of this disclosure.

FIG. 10 shows mass spectra for selected E3 ligand, Pomalidomide of this disclosure.

FIGS. 11A and 11B show H1 and C13 NMR spectra for selected POI, Palbociclib of this disclosure.

FIG. 12 shows mass spectra for selected POI, Palbociclib of this disclosure.

FIG. 13 shows mass spectra for selected DNA strands of this disclosure. Figure discloses SEQ ID NOS 1-4, 3, and 5, respectively, in order of appearance.

FIG. 14 shows mass spectra for selected DNA strands of this disclosure. Figure disclosures SEQ ID NOS 3, 6, 1 and 7, respectively, in order of appearance.

FIG. 15 shows the chemical structure of the iNH2 modifier used in this disclosure.

### DETAILED DESCRIPTION

The entire document is intended to be related as a unified disclosure, and it should be understood that all combinations of features described herein are contemplated, even if the combination of features are not found together in the same sentence, or paragraph, or section of this document. The present disclosure as illustratively described in the following may suitably be practiced in the absence of any element or elements, limitation or limitations, not specifically disclosed herein.

This disclosure provides for a chemical strategy to prompt ligand-dependent target protein degradation via chemical conjugation with a DNA-PROTAC that utilizes the function of the Cereblon E3 ubiquitin ligase complex. When an E3 ubiquitin ligase covalently attaches many ubiquitin molecules to a terminal lysine residue, the protein is marked for proteasome degradation. This process breaks the protein down into smaller peptides and ultimately its constituent amino acids, which are used to make new proteins.

DNA-PROTACs of this disclosure include E3 ligase ligand that binds to E3 Ubiquitin Ligase (typically through cereblon), and a ligand to a protein of interest (POI) (also referred to as a “targeting ligand.” DNA-PROTACs can be used for therapeutic purposes by the methods described herein. Also provided herein are compositions thereof and methods for their preparation and manufacture.

Through their recruitment to E3 ubiquitin ligase and subsequent ubiquitination, DNA-PROTACs, promoted proteasome-mediated destruction of specific proteins. These compounds, which resemble drugs, present the potential for controlling the temporal levels of selected proteins of interest. By eliminating pathogenic or oncogenic proteins, DNA-PROTACs can cause a protein of interest to become inactive when the DNA-PROTAC is added to cells or administered to an animal or human, resulting in a new paradigm for the treatment of diseases.

The human proteome comprises approximately 20,000 proteins, and it is estimated that more than 600 of them are functionally important for various types of cancer, including nearly 400 non-enzyme proteins that are challenging to target by traditional occupancy-driven pharmacology. Occupancy-driven pharmacology with small molecule inhibitors hinges on the prerequisite of target proteins having compatible binding pockets, rendering roughly 85% of the proteome “undruggable” and amplifying the risk of cumulative toxicities and drug resistance due to the requirement for high working concentrations. To overcome these challenges, a new drug discovery strategy was developed, known as Targeted Protein Degradation (TPD), which entails degrading rather than merely inhibiting proteins (Pettersson, M.; Crews, C. M. PROteolysis TArgeting Chimeras (PROTACs)—Past, Present and Future. Drug Discovery Today: Technologies. 2019. doi.org/10.1016/j.ddtec.2019.01.002; Li, X.; Pu, W.; Zheng, Q.; Ai, M.; Chen, S.; Peng, Y. Proteolysis-Targeting Chimeras (PROTACs) in Cancer Therapy. Molecular Cancer. 2022. doi.org/10.1186/s12943-021-01434-3). By co-opting protein degradation pathways, TPD facilitates complete removal of the protein molecules from within or outside the cell. While the pioneering proteolysis-targeting chimera (PROTAC) technology and molecular glues hijack the ubiquitin-proteasome system (UPS), newer modalities co-opt autophagy or the endo-lysosomal pathway. Using this mechanism, TPD is poised to expand the druggable space far beyond that amenable to small-molecule inhibitors. The event-driven mechanism of action (MOA) of TPD offers several advantages compared to traditional occupancy-driven small molecule inhibitors, such as a catalytic nature, reduced dosage, and efficacy in the face of drug-resistance mechanisms (Martin-Acosta, P.; Xiao, X. PROTACs to Address the Challenges Facing Small Molecule Inhibitors. European Journal of Medicinal Chemistry. 2021. doi.org/10.1016/j.ejmech.2020.112993; Winter, G. E.; et al., Phthalimide Conjugation as a Strategy for in Vivo Target Protein Degradation. 1. Science 2015, 348 (6241). doi.org/10.1126/science.aab1433).

A key focus of TPD is developing heterobifunctional small-molecule degraders, including PROTACs, which contain two linked moieties, one binding the protein of interest (POI) and the other binding an E3 ligase. When a PROTAC is active, the target protein ligand binds to the POI and the E3 ligase ligand binds to E3, promoting the POI and E3 to form a ternary complex through the flexible linker. At the same time, ubiquitination labels the POI with ubiquitin causing POI degradation by the proteasome ((Burslem, G. M.; Crews, C. M. Proteolysis-Targeting Chimeras as Therapeutics and Tools for Biological Discovery. Cell. 2020. doi.org/10.1016/j.cell.2019.11.031; Schapira, M.; Calabrese, M. F.; Bullock, A. N.; Crews, C. M. Targeted Protein Degradation: Expanding the Toolbox. Nat Rev Drug Discov 2019, 18 (12). doi.org/10.1038/s41573-019-0047-y).

Since the PROTACs were first developed, several critical discoveries have been made. Until now, multiple PROTAC-like molecules have entered clinical trials. However, considerable challenges remain, and several limitations impede PROTAC clinical utility such as: 1) Bioavailability: PROTACs, with a larger size (>800 Da) and high polarity, exhibit limited water solubility, hindering their passage through physiological barriers and cell membranes. 2) Side effect risk: Non-selective E3 ligase expression in both disease and normal tissues can cause severe side effects when PROTACs disperse widely. 3) E3 ligand constraints: Most PROTACs rely on CRBN or VHL ligands, constraining their efficacy due to cell-specific E3 ligase expression and resistance issues. 4) Conditional activation: The requirement to degrade the POI selectively in diseased cells while sparing normal cells poses a significant challenge for current PROTRACs due to their limited selectivity.

Nucleic-acid-based drugs have emerged as an exciting new frontier in therapeutics. This emerging class of therapeutic agents encompasses clinically available nucleic acid drugs, such as antisense oligonucleotides (ASOs), small interfering RNAs (siRNAs), aptamers, and mRNA vaccine which are presently undergoing clinical trials. These nucleic acid drugs offer compelling advantages, including low toxicity and exceptional specificity, that underscore their promise for precision medicine. Recent years have witnessed a burgeoning interest in nucleic-acid-based TPD strategies.

While reference nucleic acid-based TPD technologies focus on employing aptamers or oligonucleotides as binding motifs, they often rely on lipofectamine transfection and typically require high concentrations to achieve efficient protein degradation. However, the characteristic nature of DNA materials, i.e., high precision of Watson-Crick base-pair have not yet been sufficiently exploited for TPD. With the rapid development of nucleic acid nanotechnology and the ability to engineer DNA nanostructures, a variety of design principles have been used to realize various dimensional architectures by rationally self-assembling nanoparticle or biomolecular scaffolds such as planar tiles, origami structures, and dynamic nanomechanical systems.

Described herein is a DNA-based Programmable Proteolysis Targeting Chimera (DNA-PROTAC) with conditional activation for efficient delivery and highly specific protein degradation. This innovative technology, based on DNA nanostructures, offers key advantages over reference PROTACs, including efficient intracellular delivery, multi-targeting, conditional activation, and enhanced protein degradation. Described herein is the development of a DNA-based protein degradation system by accommodating ligands for E3 and CDK6, leading to the potent degradation of CDK4/6 proteins. This disclosure provides for an allostery-based programmable DNA platform that can be modulated for the development of conditional DNA-PROTACs. This disclosure includes description of the programmability of the DNA duplex-based protein degradation system by transitioning from DNA duplex to branched DNA nanostructures; demonstration of protein degradation variations caused by spatial distances and evaluation of the efficiency of multi-target degradation; integration of select DNA nanostructures for direct cytoplasmic delivery system with the DNA-PROTACs described herein; and demonstration of the conditional activation of protein degradation using two distinct design approaches: i) toehold mediated conditional activation and ii) allostery mediated conditional activation of DNA-PROTAC.

DNA-PROTACs can be used alone or in combination with a therapeutic agent for a particular target protein for therapeutic applications. Their compositions, modes of use, and manufacturing processes are also provided herein.

In one embodiment, the protein of interest is a protein that is not druggable in the classic sense in that it does not have a binding pocket or an active site that can be inhibited or otherwise bound, and cannot be easily allosterically controlled. In another embodiment, the protein of interest is a protein that is druggable in the classic sense. Examples of proteins of interested are provided herein.

The present application relates to DNA-PROTACs which are covalently linked to a targeted protein ligand through a chemical conjugation (e.g., click chemistry), and may optionally further comprise a linker of varying length and functionality. The present application also relates to a technology platform of bringing targeted proteins of interest to E3 ligases, for example CRBN, for ubiquitination and subsequent proteasomal degradation using the DNA-PROTACs of this disclosure.

This technology platform provides therapies based upon depression of levels of a selected protein of interest by degradation. The novel technology allows for targeted degradation to occur in a more general nature than existing methods with respect to possible targets and different cell lines or different in vivo systems.

DNA-PROTACs of the present application may offer important clinical benefits to patients, in particular for the treatment of the disease states and conditions modulated by the proteins of interest.

Without wishing to be bound by any theory, the present disclosure is believed to be based, at least in part, on the discovery that novel programmable DNA-PROTACs which degrade selected target proteins, and/or mutants thereof are useful in the treatment of diseases mediated by said proteins or mutants thereof, particularly non-small cell lung cancer, colorectal cancer, gastric cancer, liver cancer, invasive breast cancer, lung adenocarcinoma, uterine cancer, adrenal cancer, pancreatic cancer, ovarian cancer, esophageal cancer, urinary bladder cancer, endometrial cancer, prostate cancer, low-grade glioma, glioblastoma, Spitzoid cancers, soft tissue sarcoma, papillary thyroid carcinoma, head and neck squamous cell carcinoma, congenital fibrosarcoma, congenital mesoblastic nephroma, secretory breast carcinoma, mammary analogue secretory carcinoma, acute myeloid leukemia, ductal carcinoma, pulmonary neuroendocrine tumors, pheochromocytoma, and Wilms' tumor. In some embodiments, the cancer is glioblastoma.

### Certain Definitions

Technical terms are used by their common sense unless indicated otherwise. If a specific meaning is conveyed to certain terms, definitions of terms will be given in the following in the context of which the terms are used.

The singular forms “a,” “an,” and “the” may refer to plural articles unless specifically stated otherwise.

As used herein, the term “about” means±10%.

It is intended that the chemical structures depicted herein are also meant to include compounds that differ only in the presence of one or more isotopically enriched atoms. For example, compounds having the present structures except for the replacement of hydrogen by deuterium or tritium, replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range.

As used herein, the term “operably-linked” refers to the association two chemical moieties so that the function of one is affected by the other, e.g., an arrangement of elements wherein the components so described are configured so as to perform their usual function.

As used herein, the term “nucleic acid” refers to deoxyribonucleotides or ribonucleotides and polymers thereof in either single- or double-stranded form, comprising monomers (nucleotides) containing a sugar, phosphate and a base that is either a purine or pyrimidine. Unless specifically limited, the term encompasses nucleic acids containing analogues of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

As used herein, the terms “nucleotide sequence” and “nucleic acid sequence” refer to a sequence of bases (purines and/or pyrimidines) in a polymer of DNA or RNA, which can be single-stranded or double-stranded. In some embodiments, the nucleotide sequence comprises synthetic, non-natural or altered nucleotide bases, and/or backbone modifications (e.g., a modified oligomer, which can include or exclude a morpholino oligomer, phosphorodiamate morpholino oligomer or vivo-mopholino). The terms “oligo,” “oligonucleotide” and “oligomer” may be used interchangeably and refer to such sequences of purines and/or pyrimidines. The terms “modified oligos,” “modified oligonucleotides” or “modified oligomers” may be similarly used interchangeably, and refer to such sequences that contain synthetic, non-natural or altered bases and/or backbone modifications (e.g., chemical modifications to the internucleotide phosphate linkages and/or to the backbone sugar).

Modified nucleotides can include or exclude alkylated purines; alkylated pyrimidines; acylated purines; and acylated pyrimidines. These classes of pyrimidines and purines can include or exclude pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine. Backbone modifications can include or exclude chemical modifications to the phosphate linkage. The chemical modifications to the phosphate linkage can include or exclude, e.g., phosphorodiamidate, phosphorothioate (PS), N3′-phosphoramidate (NP), boranophosphate, 2′,5′-phosphodiester, amide-linked, phosphonoacetate (PACE), morpholino, peptide nucleic acid (PNA), inverted linkages (5′-5′ and 3′-3′ linkages)) and sugar modifications (e.g., 2′-O-Me, UNA, LNA).

The oligonucleotides described herein may be synthesized using solid or solution phase synthesis methods. In some embodiments, the oligonucleotides are synthesized using solid-phase phosphoramidite chemistry (U.S. Pat. No. 6,773,885, herein incorporated by reference) with automated synthesizers, herein incorporated by reference. Chemical synthesis of nucleic acids allows for the production of various forms of the nucleic acids with modified linkages, chimeric compositions, and nonstandard bases or modifying groups attached in chosen places through the nucleic acid's entire length. In some embodiments, the oligonucleotides described herein may be synthesized using enzymatic methods which can include adding single-bases via an enzyme.

Some embodiments of the invention encompass isolated or substantially purified nucleic acid compositions. As used herein an “isolated” or “purified” DNA molecule or RNA molecule refers to a DNA molecule or RNA molecule that exists apart from its native environment and is therefore not a product of nature. An isolated DNA molecule or RNA molecule may exist in a purified form or may exist in a non-native environment. In some embodiments, the non-native environment can include or exclude a transgenic host cell. In some embodiments, the terms “isolated” or “purified” includes a nucleic acid molecule which is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

By “portion” as it relates to a nucleic acid molecule, sequence or segment of the invention, is meant a sequence having at least 3 nucleotides to up to 20 nucletides, and any number of nucleotides therein.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

As used herein, the terms “sequence identity” or “identity” or “homology” in the context of two nucleic acid or polypeptide sequences makes reference to a specified percentage of residues in the two sequences that are the same when aligned for maximum correspondence over a specified comparison window, as measured by sequence comparison algorithms or by visual inspection. In some embodiments, the identity between any two nucleic acid sequences is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, 95%, 96%, 97%, 98%, or 99%.

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Another indication that nucleotide sequences are substantially identical is if two molecules hybridize to each other under stringent conditions. Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. As used herein, the term “bind(s) substantially” refers to complementary hybridization between a probe nucleic acid and a target nucleic acid and embraces minor mismatches that in some embodiments is accommodated by reducing the stringency of the hybridization media to achieve the desired detection of the target nucleic acid sequence.

The term “complementary” as used herein refers to the broad concept of complementary base pairing between two nucleic acids aligned in an antisense position in relation to each other. When a nucleotide position in both of the molecules is occupied by nucleotides normally capable of base pairing with each other, then the nucleic acids are considered to be complementary to each other at this position. Two nucleic acids are substantially complementary to each other when at least about 50%, at least about 60%, or at least about 80% of corresponding positions in each of the molecules are occupied by nucleotides which normally base pair with each other (e.g., A:T (A:U for RNA) and G:C nucleotide pairs).

As used herein, the term “derived” or “directed to” with respect to a nucleotide molecule means that the molecule has complementary sequence identity to a particular molecule of interest.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and quaternary alkylammonium salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions, such as halide, hydroxide, carboxylate, sulfate, phosphate, nitrate, lower alkyl sulfonate, and aryl sulfonate. In some embodiments, the DNA-PROTACs of this disclosure may be in a sodium, potassium, ammonium, or quaternary salt form.

The term “administer,” “administering,” or “administration” refers to implanting, absorbing, ingesting, injecting, inhaling, or otherwise introducing a compound described herein, or a composition thereof, in or on a subject.

The term “subject” as used herein refers to humans, higher non-human primates, rodents, domestic, cows, horses, pigs, sheep, dogs and cats. In one embodiment, the subject is a human.

The term “therapeutically effective amount,” in reference to treating a disease state/condition, refers to an amount of a therapeutic agent that is capable of having any detectable, positive effect on any symptom, aspect, or characteristics of a disease state/condition when administered as a single dose or in multiple doses. Such effect need not be absolute to be beneficial.

In certain embodiments, the therapeutically effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of the targeted protein of interest in a selected tissue. In certain embodiments, the effective amount is an amount effective for promoting the degradation of a targeted protein of interest in a selected tissue by a range between a 10% to 99%, inclusive.

The terms “treat’ and “treatment” refer to both therapeutic treatment and prophylactic or preventative measures, wherein the object is to prevent or decrease an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The terms “inhibiting” or “reducing” or any variation of these terms includes any measurable decrease or complete inhibition to achieve a desired result. The terms “promote” or “increase” or any variation of these terms includes any measurable increase or production of a protein or molecule to achieve a desired result.

The term “preventing” or any variation of this term means to slow, stop, or reverse progression toward a result. The prevention may be any slowing of the progression toward the result.

As used herein, the term “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological angiogenesis as in proliferative retinopathy and tumor metastasis; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological proliferation of normally quiescent cells. In some embodiments, the proliferative diseases include cancers (i.e., “malignant neoplasms”), benign neoplasms, angiogenesis, inflammatory diseases, and autoimmune diseases.

As used herein, the term “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

The tumor microenvironment is an important aspect of cancer biology that contributes to tumor initiation, tumor progression and responses to therapy. The tumor microenvironment is composed of a heterogeneous cell population that includes malignant cells and cells that support tumor proliferation, invasion, and metastatic potential though extensive crosstalk.

In some embodiments, the cancer for which DNA-PROTACs of this disclosure can treat can include or exclude: lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer which the DNA-PROTACs of this disclosure can treat is glioblastoma. In some embodiments, the DNA-PROTACs of this disclosure can kill a cancer cell, wherein the cancer is any of the aforementioned cancers. In some embodiments, the DNA-PROTACs of this disclosure can kill a glioblastoma cancer cell.

### Programmable DNA-PROTACs

For the purpose of the present disclosure, the terms “DNA-PROTAC” and “programmable DNA-PROTAC” and “DNA proteolysis target chimeras” are used interchangeably.

In some embodiments, the present disclosure provides for a DNA-PROTAC which include a ligand targeting a protein of interest (POI), and a ligand targeting an E3 ligase, and a double-stranded DNA segment. Each of the ligands may be connected to the DNA segment by conjugation to opposing DNA strands (a “first DNA strand” and a “second DNA strand,” respectively). The connection may occur through a variety of bioconjugation chemistries, including click chemistry, strained polycyclic click chemistry, and the like. In some embodiments, the connection may occur through a spacer selected from an alkyl chain or a PEG (polyethylene glycol) chain.

In some embodiments, the present disclosure provides a programmable DNA proteolysis target chimera complex (DNA-PROTAC) comprising: a first DNA strand comprising one or a plurality of independent E3 ligase ligands; and a second DNA strand comprising one or a plurality of independent protein of interest (POI) targeting ligands. At least a portion of the first DNA strand is complementary to a portion of the second DNA strand and the first and second DNA strands form a DNA duplex. The one or a plurality of independent E3 ligase ligands is connected to the first DNA strand at one or a plurality of independently selected positions on said first DNA strand. The one or a plurality of independent POI targeting ligands is connected to the second DNA strand at one or a plurality of independently selected positions on said second DNA strand.

In some embodiments, the DNA-PROTAC can further comprise a targeting moiety. The targeting moiety can be selected from a cell-penetrating peptide or a blood-brain barrier traversal agent. The blood-brain barrier traversal agent can be a lipid, a neutral amino acid, a hormone, a vitamin, a cholesterol derivative (hydroxyl or reverse acetyl esters at each of the hydroxyl moieties on said cholesterol). The vitamin can be vitamin B, vitamin D, or vitamin E. The hormone can be Cortisol, Aldosterone, DHEA, an androgens, Adrenaline (epinephrine), Noradrenaline, Estrogen, Progesterone, or Testosterone. The lipid can be a C₆-C₁₈ fatty acid, or glycerol conjugate thereof. The cell-penetrating peptide can be any of the cell-penetrating peptides listed in U.S. Pat. Nos. 10,288,601; 10,967,000; 10,626,147; 10,300,118; 10,253,099; 10,421,784; and 9,303,076, each of which is herein incorporated by reference.

To economize degradation while in the presence of a E3 ligase, in some embodiments the DNA-PROTAC can include two or more POI targeting ligands which target different proteins. The two or more POI targeting ligands can be the same, or different. The DNA-PROTAC can recruit two separate proteins to be marked for degradation by the E3 ligase to disrupt or reduce the levels of two or more different proteins. When two different proteins are degraded by the same DNA-PROTAC, multiple pathways can be reduced or inhibited, resulting in a synergistic effect when disrupting cellular function of a target cell, e.g., a proliferative cell (and in particular, a cancer cell).

In some embodiments, to increase avidity to a target protein of interest, the DNA-PROTAC can comprise two or more targeting ligands to the same protein of interest. The targeting ligands can be the same or different. When the targeting ligands are the same, affinity is enhanced. When the targeting ligands are different, avidity is enhanced. The different targeting ligands can be configured to bind to different portions of the same protein.

In some embodiments, to increase the affinity of recruiting an E3 ligase, the DNA-PROTAC can comprise two or more E3 ligase ligands. When a protein of interest is bound to the DNA-PROTAC which includes at least two bound E3 ligases, the statistical rate of degradation of the protein can be increased, resulting in a faster rate of overall protein degradation.

The one or plurality of independently selected positions on the first or second DNA strand where the E3 ligase or POI targeting ligand is positioned can be configured to vary in space and angle. Use of a double-stranded DNA helix, both the distance and orientation between the E3 ligase ligand and the POI targeting ligand can be controlled. Furthermore, use of a Holliday junction can enforce structural rigidity to ensure the positions between the E3 ligase ligand site and the POI targeting ligand site are structurally rigidly separated (Chem. Soc. Rev., 2021, 50, 11966-11978, doi.org/10.1039/D1CS00250C). Moreover, the design of the DNA double-stranded sequence can be selected to modulate the persistence length of the double-strand, which further maintains the rigidity of the helix to maintain the POI targeting ligand and E3 ligase ligand positions.

In some embodiments, the selected position on the first DNA strand and the selected position on the second DNA strand can be separated by a distance ranging from about 0.99 nm (99 Angstrom) to about 7 nm, inclusive. In some embodiments, the selected position on the first DNA strand and the selected position on the second DNA strand can be separated by a rotational angle about the double-stranded DNA complex ranging from about 36 to about 180 degrees, inclusive. In some embodiments, the selected position on the first DNA strand and the selected position on the second DNA strand can be separated by a distance of about a minor groove to about a major groove.

In some embodiments, the sequences of the DNA strands can be modified to include a nuclease resistance feature. Avoiding premature nuclease degradation increases the circulating half-life of the DNA-PROTACs post-administration to a subject. The nuclease resistance feature can be a sugar modification or an internucleoside linkage modification. The sugar modification can be a locked nucleic acid, a threose nucleic acid, or a 2′-alkoxy modification. The internucleoside linkage modification can be a phosphorothioate, phosphoroselenoate, or phosphoramidate.

As used herein, the terms “targeting ligand” and “protein targeting ligand” and “protein of interest (POI) targeting ligand” are using interchangeably and are to be construed to encompass any molecules ranging from small molecules to large proteins that associate with or bind to a protein of interest. In certain embodiments, the targeting ligand and respective targeted protein of interest are set forth in Table 1, and include CDK6, CDK4, BCR-Abl, EGFR, BTK, BRD4, HDAC6, STAT3, BCL-XI, FAK, P38-alpha, myc, Arora, Ras, and Jak as target proteins of interest. In certain embodiments, the target protein of interest is a mutant of CDK6, CDK4, BCR-Abl, EGFR, BTK, BRD4, HDAC6, STAT3, BCL-XI, FAK, P38-alpha, myc, Arora, Ras, and Jak.

In one embodiment, a mutant of a selected target protein of interest can include or exclude a: translocation, deletion, or inversion event which causes or is caused by a medical disorder. In some embodiments, the mutation of a selected target protein of interest can include or exclude a post-translational modification selected from: phosphorylation, acetylation, acylation including propionylation and crotylation, N-linked glycosylation, O-linked glycosylation, amidation, hydroxylation, methylation and poly-methylation, pyrogultamoylation, myristoylation, farnesylation, geranylgeranylation, ubiquitination, sumoylation, sulfation, and combinations thereof. In some embodiments, the post-translational modification is caused by a medical disorder.

In some embodiments, the target protein of interest is a mutant protein found in cancer cells, or a protein, for example, where a partial, or full, gain-of-function or loss-of-function is encoded by nucleotide polymorphisms. In some embodiments, the protein targeting ligand targets the aberrant form of the protein and not the normal form of the protein.

In some embodiments, the target binding ligand is a ligand, drug, antibody, aptamer, scFv, or nanobody which preferentially binds to a target protein of interest. In some embodiments, the target binding ligand is a target binding ligand listed in Table 1. In some embodiments, the target binding ligand is selected from: paldocicib, GNF-5, Gefitinib, Ibrutinib, OTX-015, SD-36, ABT-263, and foretinib, and variants thereof. In some embodiments, the target binding ligand is a HDAC6 inhibitor. In some embodiments, the HDAC6 inhibitor is selected from: Vorinostat, Romidepsin, Panobinostat, and Belinostat. In some embodiments, the HDAC6 inhibitor is selected from: CAY10603, WT161, ACY-738, KA2507, Citarinostat (ACY-241), Tubacin, Ricolinostat (ACY-1215), Nexturastat A, ACY-775, Tubastatin A HCl, Tubastatin A TFA, Tubastatin A, HPOB, and SKLB-23bb (all available from Selleck Chem, USA). In some embodiments, the target binding ligand is a BRD4 inhibitor. The BRD4 inhibitor can include the BRD4 inhibitors provided in U.S. Pat. No. 10,646,575, herein incorporated by reference. In some embodiments, the BRD4 inhibitor is selecte from: BRD4770, BRD4 Inhibitor-10, FL-411, BI 2536, I-BET151 (GSK1210151A), PFI-1 (PF-6405761), (+)-JQ1, Bromosporine, SGC-CBP30, CPI-203, MS436, Birabresib (OTX015), XMD8-92, GSK1324726A (I-BET726), I-BRD9, Pelabresib (CPI-0610), Mivebresib (ABBV-075), AZD5153 6-hydroxy-2-naphthoic acid, F2523, ABBV-744, ZL0420, INCB054329, dBET6, dBET1, PLX51107, ARV-825, A1874, SRX3207, dBET57, Y06036, ARV-771, MZ-1, GSK778, GSK046, (R)-(−)-JQ1 Enantiomer, GNE-781, Thalidomide-NH—C4-NH-Boc, and NHWD-870 (all available from Selleck Chem, USA). In some embodiments, the target binding ligand is a CDK6 inhibitor. The CDK6 inhibitor can include or exclude: Abemaciclib, Palbociclib, and Ribociclib. In some embodiments, the target binding ligand is a EGFR inhibitor. The EGFR inhibitor can include or exclude: erlotinib, osimertinib, neratinib, gefitinib, dacomitinib, lapatinib, mobocertinib, and vandetanib. In some embodiments, the target binding ligand is a BCR-Abl inhibitor. The BCR-Abl inhibitor can include or exclude: imatinib, nilotinib, dasatinib, bosutinib, ponatinib, asciminib, and dasatinib. In some embodiments, the target binding ligand is a BTK inhibitor. The BTK inhibitor can include or exclude: ibrutinib, acalabrutinib, and zanubrutinib. In some embodiments, the target binding ligand is a STAT3 inhibitor. The STAT3 inhibitor can include or exclude: HJC0152, Cucurbitacin I, Cucurbitacin IIb, APTSTAT3-9R, SC-1, SC99, Brevilin A, Scutellarin, GYY4137, C188-9, Niclosamide, STAT3-IN-1, WP1066, Cryptotanshinone (Tanshinone C), Stattic, inS3-54-A18, Resveratrol, Morusin, NSC 74859 (S31-201), Kaempferol-3-O-rutinoside, Ochromycinone (STA-21) Colivelin Ginkgolic acid C17:1, HO-3867, Napabucasin (BBI608), Artesunate (WR-256283), Bosutinib, and TPCA-1, SC-43 (all of which are available from Selleck Chem, USA). In some embodiments, the STAT3 inhibitor is selected from: IMX-110, AZD9150, Napabucasin, Bazedoxifene, Siltuximab, CNTO 328, Ruxolitinib, Itacitinib, Ponatinib, and Sunitinib. In some embodiments, the target binding ligand is a BCI-XL inhibitor. The BCI-XI inhibitor can include or exclude: ABT-263/“Navitoclax,” and A-1331852. In some embodiments, the target binding ligand is a FAK inhibitor. The FAK inhibitor can include or exclude: TAE226 (NVP-226), VS-6062 (PF00562271), PF-573228 (PF-228), VS-6063 (Defactinib), GSK2256098, VS-4718 (PND-1186), Y15, C4, R2, BI 853520 (IN10018, ifebemtinib), CT-707 (Conteltinib), AMP-945 (Narmafotinib), and APG-2449. In some embodiments, the target binding ligand is a P38-alpha inhibitor. The P38-alpha inhibitor can include or exclude: PH 797804, DBM 1285 dihydrochloride, SB 706504, AL 8697, TAK 715, AMG 548, VX 745, SB 202190, SB 203580, BIRB 796, SB 203580 hydrochloride, SB 239063, EO 1428, RWJ 67657, and SCIO 469 hydrochloride (all of which are available from R&D Systems, USA). In some embodiments, the P38-alpha inhibitors can include or exclude: ralimetinib and foretinib. In some embodiments, the P38-alpha inhibitors can be those identified in Smith, et al. (Nature Communications, 10, 131 (2019), doi.org/10.1038/s41467-018-08027-7), herein incorporated by reference. In some embodiments, the variants are conjugates of the aforementioned compounds through reaction of a phenol, alcohol, amide, alkynyl, amino, carboxy, or acrylamido functional moiety on said compounds.

In some embodiments, targeting ligands also include their pharmaceutically acceptable salts, prodrugs and isotopic derivatives.

In some embodiments, the targeted protein of interest mediates chromatin structure and function. The targeted protein of interest may mediate an epigenetic action such as DNA methylation or covalent modification of histones. An example is histone deacetylase (HDAC6).

In some embodiments, the targeted protein of interest mediates mitotic cell cycle, including cell division, which includes cell division protein kinase 6 (CDK6). Cyclin-CDK dual complexes are the major driving machinery for cell cycle progression. Among the CDKs, Cyclin-dependent kinases 4 and 6 (CDK4/6) are key orchestrators of cell cycle regulation as they control the progression from G1- to S-phase of the cell cycle. Dysregulation of the cell cycle, fueled by up regulated and over-activated CDKs, significantly contributes to uncontrolled cell proliferation, which is a fundamental hallmark of cancer. The CDK4/6 inhibitors have been approved by FDA for the treatment of patients advanced or metastatic breast cancer.

In some embodiments, the targeted protein of interest mediates cellular adhesion, mitogenic activiation, or apoptosis inhibition (BCR-Abl).

In some embodiments, the targeted protein of interest mediates cell proliferation, invation, metastasis, apoptosis, and angiogenesis (EGFR).

In some embodiments, the targeted protein of interest mediates B-cell development (BTK). In some embodiments, the targeted protein of interest mediates tumor proliferation, metastasis, and invasion.

In some embodiments, the targeted protein of interest is a reader of lysine acetylation (BRD4).

In some embodiments, the targeted protein of interest mediates cell growth, and apoptosis (STAT3).

In some embodiments, the targeted protein of interest mediates cell apoptosis (BCI-XI).

In some embodiments, the targeted protein of interest mediates cell growth (FAK).

In some embodiments, the targeted protein of interest mediates proliferation, differentiation, and transcription regulation (P38-alpha).

In some embodiments, the targeted protein of interest is a modulator of a signaling cascade related to a known disease state. In another embodiment, the targeted protein of interest mediates a disorder by a mechanism different from modulating a signaling cascade. Any protein in a eukaryotic system or a microbial system are targets for proteasomal degradation using the present invention. In some embodiments, the targeted protein of interest may be a eukaryotic protein (e.g., a human protein).

The term “E3 ligase ligand” or “ligand for E3 ligase” or “E3-ligase binding ligand” refers to a compound targeting an E3 ligase. In certain embodiments, the E3 ligase ligand comprises one or more of cereblon E3 ligase, a VHL E3 ligase, a MDM2 ligase, a TRIM24 ligase, a TRIM21 ligase, a KEAP1 ligase, and an IAP ligase. In certain embodiments, the E3 ligase ligand is selected from: pomalidomide, thalidomide, lenalidomide, VH032, adamantane, 1-((4, 4, 5, 5, 5-pentafluoropentyl) sulfinyl) nonane, nutlin-3a, RG7112, RG7338, AMG 232, AA-115, bestatin, MV1, LCL161, and/or analogs thereof. In some embodiments, the E3-ligase binding ligand include those listed in Table 1.

The term “DNA strand” as used in this disclosure refers to a hybridizable nucleic acid strand which comprises at least three nucleic acids. In some embodiments, the DNA strands of this disclosure can have a sequence of any of SEQ ID NOs: 1-16. In some embodiments, the DNA strands of this disclosure can have a sequence having at least about 60% sequence identity to any one of SEQ ID NO:1-16. In some embodiments, the DNA strands of this disclosure comprises a nucleic acid sequence having at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO: 1-16. In some embodiments, the DNA strands of this disclosure consists of a nucleic acid sequence having at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO: 1-16. In certain embodiments, the DNA strands of this disclosure comprises any sequence comprising any one of SEQ ID NO: 1-16.

In some embodiments, the DNA strands of this disclosure comprises one or more modified nucleic acids. In some embodiments, the one or more modified nucleic acids are alkynyl-modified nucleotides.

In some embodiments, the alkynyl modified nucleotides are chemically synthesized from a phosphoramidite selected from: 5′-Dimethoxytrityl-5-[(6-oxo-6-(dibenzo[b,f]azacyclooct-4-yn-1-yl)-capramido-N-hex-6-yl)-3-acrylimido]-2′-deoxyUridine, 3′-[(2-cyanoethyl)-(N,N-diisopropyl)]-phosphoramidite, 5′-Dimethoxytrityl-5-ethynyl-2′-deoxyUridine, 3′-[(2-cyanoethyl)-(N,N-diisopropyl)]-phosphoramidite, 5′-Hexynyl Phosphoramidite, 5′-Dimethoxytrityl-5-(octa-1,7-diynyl)-2′-deoxyuridine, 3′-[(2-cyanoethyl)-(N,N-diisopropyl)]-phosphoramidite, 10-(6-oxo-6-(dibenzo[b,f]azacyclooct-4-yn-1-yl)-capramido-N-ethyl)-O-triethyleneglycol-1-[(2-cyanoethyl)-(N,N-diisopropyl)]-phosphoramidite, 6-Bromo-hex-1-yl-(2-cyanoethyl)-(N,N-diisopropyl)-phosphoramidite, 3-Dimethoxytrityloxy-2-(3-(5-hexynamido) propanamido) propyl-1-O-[(2-cyanoethyl)-(N,N-diisopropyl)]-phosphoramidite, and 5′-Dimethoxytrityl-3′-propargyl-5-methyl-2′-deoxyCytosine-N-succinoyl-long chain alkylamino-CPG.

As used herein, the term “therapeutic agent” refers to agents that provide a therapeutically desirable effect when administered to an animal. The animal is a mammal, which can include or exclude a human. The therapeutic agent may be of natural or synthetic origin. In some embodiments, the therapeutic agent can include or exclude a nucleic acid, a polypeptide, a protein, a peptide, a radioisotope, saccharide or polysaccharide or an organic compound, which can include or exclude a small molecule. The term “small molecule” includes organic molecules having a molecular weight of less than about, e.g., 1000 daltons. In one embodiment a small molecule can have a molecular weight of less than about 800 daltons. In another embodiment a small molecule can have a molecular weight of less than about 500 daltons.

TABLE 1
Target proteins and corresponding representative target ligands which can be
conjugated at the amino-, phenolic, alkynyl, acrylamidyl, carboxyl, or alcoholic sites to
connect to a DNA strand for forming a DNA-PROTAC of this disclosure.

| | | | | E3-ligase |
|---|---|---|---|---|
| Target | | | | binding |
| Protein | Gene ID | Indication | Target binding ligand | ligand |
| CDK6 | 1021 | mitotic cell cycle; cell divison protein kinase 6 gene | 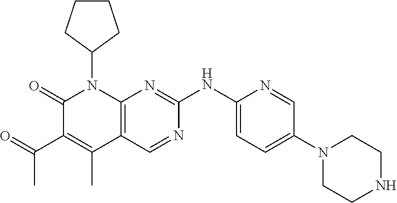 [SMILES: CC(=O)c1c(C)c2cnc(Nc3ccc(N4CCNCC4)cn3)nc2n(C2CCCC2)c1=O] | CRBN |
| | | | Palbociclib | |
| BCR- Abl | 107963955 | cellular adhesion; mitogenic activation; apoptosis inhibition | 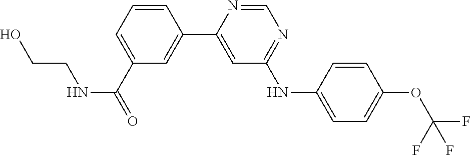 [SMILES: O=C(NCCO)c1cccc(-c2cc(Nc3ccc(OC(F)(F)F)cc3)ncn2)c1] | VHL |
| | | | GNF-5 | |
| EGFR | 1956 | cell proliferation, invasion, metastasis, apoptosis, and angiogenesis | 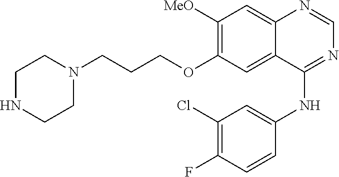 [SMILES: COc1cc2ncnc(Nc3ccc(F)c(Cl)c3)c2cc1OCCCN1CCNCC1] | CRBN |
| | | | Gefitinib | |
| BTK | 695 | B-cell development | 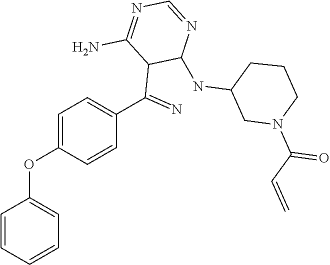 [SMILES: C=CC(=O)N1CCCC(NC2N=CN=C(N)C2C(=N)c2ccc(Oc3ccccc3)cc2)C1] | CRBN |
| | | | Ibrutinib | |
| BRD4 | 23476 | tumor proliferation, metastasis, and invasion | 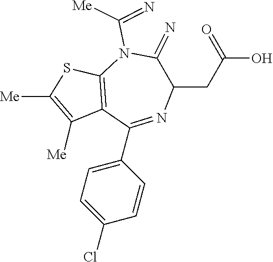 [SMILES: CC(=N)N1C(=N)C(CC(=O)O)N=C(c2ccc(Cl)cc2)c2c1sc(C)c2C] | CRBN |
| | | | OTX-015 | |
| HDAC6 | 10013 | transcriptional regulation, cell cycle progression | 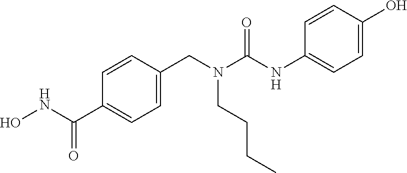 [SMILES: CCCCN(Cc1ccc(C(=O)NO)cc1)C(=O)Nc1ccc(O)cc1] | CRBN |
| | | | HDAC6 inhibitor | |
| STAT3 | 6774 | cell growth, apoptosis | 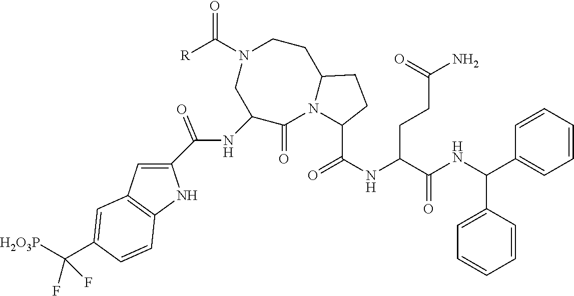 | CRBN |
| | | | SD-36 | |
| BCL-XI | 598 | cell apoptosis | 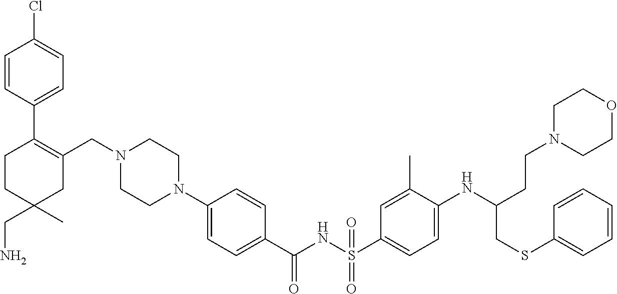 [SMILES: Cc1cc(S(=O)(=O)NC(=O)c2ccc(N3CCN(CC4=C(c5ccc(Cl)cc5)CCC(C)(CN)C4)CC3)cc2)ccc1NC(CCN1CCOCC1)CSc1ccccc1] | CRBN |
| | | | ABT-263 | |
| FAK | 5747 | cell growth | 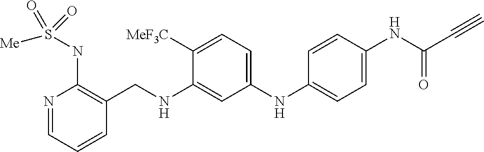 | CRBN |
| | | | FAK inhibitor | |
| P38a | 1432 | proliferation, differentia- tion, transcription regulation | 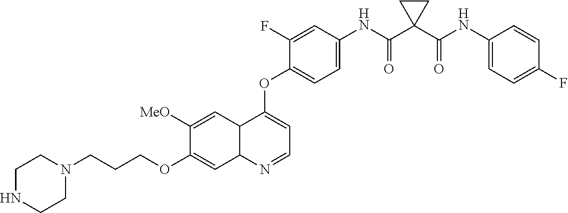 [SMILES: COC1=CC2C(Oc3ccc(NC(=O)C4(C(=O)Nc5ccc(F)cc5)CC4)cc3F)=CC=NC2C=C1OCCCN1CCNCC1] | CRBN |
| | | | Foretinib | |

### Certain Methods

In some embodiments, this disclosure provides a method for identifying a DNA-PROTAC which mediates degradation or reduction of protein of interest, the method comprising: providing a test DNA-PROTAC comprising an ligand to a selected POI, conjugated to a first DNA strand, wherein the first DNA strand is hybridized to a second DNA strand which is connected to a E3 ligase ligand; contacting the test DNA-PROTAC with a cell comprising a E3 ligase and the protein of interest; determining whether the level of the protein of interest is decreased in the cell; and identifying the test DNA-PROTAC as a DNA-PROTAC which mediates degradation or reduction of the selected protein of interest. In some embodiments, the method for identifying a DNA-PROTAC which mediates degradation or reduction of protein of interest is performed in the presence of a proteasome inhibitor to confirm that the mechanism of action is by proteolysis of the target protein of interest (when the protein of interest is not reduced when in the presence of the proteasome inhibitor). In certain embodiments, the cell is a cancer cell. In certain embodiments, the cancer cell is a glioblastoma cancer cell.

In some embodiments, a DNA-PROTAC of the present disclosure is more efficacious in treating a disease or condition (e.g., cancer) than the targeting ligand alone, or without linkage to an E3 ligase ligand. In some embodiments, the DNA-PROTAC of the present disclosure that is more efficacious in treating a disease or condition than, or is capable of treating a disease or condition resistant to, the targeting ligand, is more potent in inhibiting the growth of cells (e.g., cancer cells) or decreasing the viability of cells (e.g., cancer cells), than the targeting ligand alone. In certain embodiments, the DNA-PROTAC inhibits the growth of cells (e.g., cancer cells) or decreases the viability of cells (e.g., cancer cells) at an IC50 that is lower than the IC50 of the targeting ligand for inhibiting the growth or decreasing the viability of the cells. In certain embodiments, the IC50 of the DNA-PROTAC is at most 90%, 80%, 70%, 60%, 50%, 40%, 30%, 20%, 10%, 8%, 5%, 4%, 3%, 2%, 1%, 0.8%, 0.5%, 0.4%, 0.3%, 0.2%, or 0.1% of the IC50 of the targeting ligand alone. In certain embodiments, the IC50 of the DNA-PROTAC is at most 50%, 40%, 30%, 20%, 10%, 8%, 5%, 4%, 3%, 2%, 1%, 0.8%, 0.5%, 0.4%, 0.3%, 0.2%, or 0.1% of the IC50 of the targeting ligand alone. In certain embodiments, the IC50 of the DNA-PROTAC is at most 30%, 20%, 10%, 8%, 5%, 4%, 3%, 2%, 1%, 0.8%, 0.5%, 0.4%, 0.3%, 0.2%, or 0.1% of the IC50 of the targeting ligand alone. In certain embodiments, the IC50 of the DNA-PROTAC is at most 10%, 8%, 5%, 4%, 3%, 2%, 1%, 0.8%, 0.5%, 0.4%, 0.3%, 0.2%, or 0.1% of the IC50 of the targeting ligand alone. In certain embodiments, the IC50 of the DNA-PROTAC is at most 5%, 4%, 3%, 2%, 1%, 0.8%, 0.5%, 0.4%, 0.3%, 0.2%, or 0.1% of the IC50 of the targeting ligand alone. In certain embodiments, the IC50 of the DNA-PROTAC is at most 2%, 1%, 0.8%, 0.5%, 0.4%, 0.3%, 0.2%, or 0.1% of the IC50 of the targeting ligand alone. In certain embodiments, the IC50 of the DNA-PROTAC is at most 1%, 0.8%, 0.5%, 0.4%, 0.3%, 0.2%, or 0.1% of the IC50 of the targeting ligand alone. In certain embodiments, the DNA-PROTAC inhibits the growth of cells (e.g., cancer cells) or decreases the viability of cells (e.g., cancer cells) at an Emax that is lower than the Emax of the targeting ligand alone for inhibiting the growth or decreasing the viability of the cells. In certain embodiments, the Emax of the DNA-PROTAC is at most 90%, 80%, 70%, 60%, 50%, 40%, 30%, 20%, 10%, 8%, 5%, 4%, 3%, 2%, or 1% of the Emax of the targeting ligand alone. In certain embodiments, the Emax of the DNA-PROTAC is at most 50%, 40%, 30%, 20%, 10%, 8%, 5%, 4%, 3%, 2%, or 1% of the Emax of the targeting ligand alone. In certain embodiments, the Emax of the DNA-PROTAC is at most 90%, 80%, 70%, 60%, 50%, 40%, 30%, 20%, or 10% of the Emax of the targeting ligand alone. In certain embodiments, the Emax of the DNA-PROTAC is at most 90%, 80%, 70%, 60%, 50%, 40%, or 30% of the Emax of the targeting ligand alone.

In some embodiments, the DNA-PROTACs of the present application that is more efficacious in treating a disease or condition than, or is capable of treating a disease or condition resistant to, the targeting ligand alone, wherein the disease or condition is cancer (e.g., cancer described herein). In further embodiments, the cancer is glioblastoma.

In some embodiments, the DNA-PROTACs of this disclosure promotes the degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of a targeted protein of interest at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In certain embodiments, the DNA-PROTACs of this disclosure increases the rate of a targeted protein of interest degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

Certain embodiments of the invention also provide a method of treating a disease or disorder in a subject, comprising administering to the subject a therapeutically effective amount of a composition as described herein.

In some embodiments, a method of the invention further comprises administering at least one therapeutic agent to the subject. In some embodiments, the at least one therapeutic agent is administered in combination with the DNA-PROTAC. As used herein, the phrase “in combination” refers to the simultaneous or sequential administration of the DNA-PROTAC and the at least one therapeutic agent. For simultaneous administration, the DNA-PROTAC and the at least one therapeutic agent is present in a single composition or is separate. In some embodiments, when the DNA-PROTAC and at least one therapeutic agent are administered simultaneously, they are administered by either the same or different routes.

In some embodiments, this disclosure provides a method of treating a disease or disorder in a subject, comprising administering to the subject a therapeutically effective amount of a DNA-PROTAC or composition comprising a DNA-PROTAC as described herein.

In some embodiments, the disease or disorder is cancer.

In some embodiments, the cancer is glioblastoma.

In some embodiments, the method further comprises administering at least one therapeutic agent to the subject.

In some embodiments, the therapeutic agent is a chemotherapeutic drug. In some embodiments, the chemotherapeutic drug is selected from: Abraxane (chemical name: albumin-bound or nab-paclitaxel), Adriamycin (chemical name: doxorubicin), carboplatin (brand name: Paraplatin), Cytoxan (chemical name: cyclophosphamide), daunorubicin (brand names: Cerubidine, DaunoXome), Doxil (chemical name: doxorubicin), Ellence (chemical name: epirubicin), fluorouracil (also called 5-fluorouracil or 5-FU; brand name: Adrucil), Gemzar (chemical name: gemcitabine), Halaven (chemical name: eribulin), Ixempra (chemical name: ixabepilone), methotrexate (brand names: Amethopterin, Mexate, Folex), Mitomycin (chemical name: mutamycin), mitoxantrone (brand name: Novantrone), Navelbine (chemical name: vinorelbine), Taxol (chemical name: paclitaxel), Taxotere (chemical name: docetaxel), thiotepa (brand name: Thioplex), vincristine (brand names: Oncovin, Vincasar PES, Vincrex), and Xeloda (chemical name: capecitabine). In some embodiments, the chemotherapeutic agent is selected from: Abraxane (Paclitaxel (with albumin) Injection), Adriamycin (Doxorubicin), Afinitor (Everolimus), Alecensa (Alectinib), Alimta (PEMETREXED), Aliqopa (Copanlisib), Alkeran Injection (Melphalan), Alunbrig (Brigatinib), Aredia (Pamidronate), Arimidex (Anastrozole), Aromasin (Exemestane), Arranon (Nelarabine), Arzerra (Ofatumumab), Avastin (Bevacizumab), Bavencio (Avelumab), Beleodaq (Belinostat), Besponsa (Inotuzumab Ozogamicin), Bexxar (Tositumomab), BiCNU (Carmustine), Blenoxane (Bleomycin), Blincyto (Blinatumomab), Bosulif (Bosutinib), Braftovi (Encorafenib), Busulfex (Busulfan), Cabometyx (Cabozantinib), Calquence (Acalabrutinib), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Cinqair (Reslizumab), Clolar (Clofarabine), Cometriq (Cabozantinib), Copiktra (Duvelisib), Cosmegen (Dactinomycin), Cotellic (Cobimetinib), Cyramza (Ramucirumab), CytosarU (Cytarabine), Cytoxan (Cytoxan), Cyclophosphamide, Dacogen (Decitabine), Darzalex (Daratumumab), DaunoXome (Daunorubicin Lipid Complex), Daurismo (Glasdegib), Decadron (Dexamethasone), DepoCyt (Cytarabine Lipid Complex), Dexamethasone Intensol (Dexamethasone), Dexpak Taperpak (Dexamethasone), Docefrez (Docetaxel), Doxil (Doxorubicin Lipid Complex), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Emend (Fosaprepitant), Empliciti (Elotzumab), Erbitux (Cetuximab), Erivedge (Vismodegib), Erleada (Apalutamide), Erwinaze (Asparaginase Erwinia chrysanthemi), Ethyol (Amifostine), Etopophos (Etoposide), Eulexin (Flutamide), Fareston (Toremifene), Farydak (Panobinostat), Faslodex (Fulvestrant), Femara (Letrozole), Firmagon (Degarelix), FloPred (Prednisolone), Fludara (Fludarabine), Folex (Methotrexate), Folotyn (Pralatrexate), FUDR (FUDR (floxuridine)), Gazyva (Obinutuzumab), Gemzar (Gemcitabine), Gilotrif (Afatinib), Gleevec (Imatinib Mesylate), Halaven (Eribulin), Herceptin (Trastuzumab), Hexalen (Altretamine), Hycamtin (Topotecan), Hycamtin (Topotecan), Hydrea (Hydroxyurea), Ibrance (Palbociclib), Iclusig (Ponatinib), Idamycin PFS (Idarubicin), Idhifa (Enasidenib), Ifex (Ifosfamide), Imbruvica (Ibrutinib), Imfinzi (Durvalumab), Imlygic (Talimogene Laherparepvec), Inlyta (Axitinib), Intron A alfab (Interferon alfa-2a), Iressa (Gefitinib), Istodax (Romidepsin), Ixempra (Ixabepilone), Jakafi (Ruxolitinib), Jevtana (Cabazitaxel), Kadcyla (Ado-trastuzumab Emtansine), Keytruda (Pembrolizumab), Kisqali (Ribociclib), Kyprolis (Carfilzomib), Lanvima (Lenvatinib), Leukeran (Chlorambucil), Leukine (Sargramostim), Leustatin (Cladribine), Lorbrena (Lorlatinib), Lupron (Leuprolide), Lynparza (Olaparib), Lysodren (Mitotane), Matulane (Procarbazine), Megace (Megestrol), Mekinist (Trametinib), Mektovi (Binimetinib), Mesnex (Mesna), Mustargen (Mechlorethamine), Mutamycin (Mitomycin), Myleran (Busulfan), Mylotarg (Gemtuzumab Ozogamicin), Navelbine (Vinorelbine), Nerlynx (Neratinib), Neulasta (filgrastim), Neulasta (pegfilgrastim), Neupogen (filgrastim), Nexavar (Sorafenib), Nilandron (Nilandron (nilutamide)), Ninlaro (Ixazomib), Nipent (Pentostatin), Nolvadex (Tamoxifen), Odomzo (Sonidegib), Oncaspar (Pegaspargase), Oncovin (Vincristine), Opdivo (Nivolumab), Panretin (Alitretinoin), Paraplatin (Carboplatin), Perjeta (Pertuzumab), Platinol (Cisplatin), PlatinoIAQ (Cisplatin), Pomalyst (Pomalidomide), Portrazza (Necitumumab), Proleukin (Aldesleukin), Purinethol (Mercaptopurine), Reclast (Zoledronic acid), Revlimid (Lenalidomide), Rituxan (Rituximab), RoferonA alfaa (Interferon alfa-2a), Rubex (Doxorubicin), Rubraca (Rucaparib), Rydapt (Midostaurin), Sandostatin (Octreotide), Soltamox (Tamoxifen), Sprycel (Dasatinib), Stivarga (Regorafenib), Sutent (Sunitinib), Sylvant (Siltuximab), Synribo (Omacetaxine), Tabloid (Thioguanine), Taflinar (Dabrafenib), Tagrisso (Osimertinib), Talzenna (Talazoparib), Tarceva (Erlotinib), Targretin Capsules (Bexarotene), Tasigna (Decarbazine), Taxol (Paclitaxel), Taxotere (Docetaxel), Tecentriq (Atezolizumab), Temodar (Temozolomide), Tepadina (Thiotepa), Thioplex (Thiotepa), Tibsovo (Ivosidenib), Toposar (Etoposide), Torisel (Temsirolimus), Treanda (Bendamustine hydrochloride), Trelstar (Triptorelin), Tykerb (lapatinib), Unituxin (Dinutuximab), Valstar (Valrubicin), Varubi (Rolapitant), Vectibix (Panitumumab), Velban (Vinblastine), Velcade (Bortezomib), Venclexta (Venetoclax), Vepesid (Etoposide), Vepesid (Etoposide Injection), Verzenio (Abemaciclib), Vesanoid (Tretinoin), Vidaza (Azacitidine), Vincasar PFS (Vincristine), Vincrex (Vincristine), Vistogard (Uridine Triacetate), Vitrakvil (Larotrectinib), Vizimpro (Dacomitinib), Votrient (Pazopanib), Vumon (Teniposide), Wellcovorin IV (Leucovorin), Xalkori (Crizotinib), Xeloda (Capecitabine), Xospata (Gilteritinib), Xtandi (Enzalutamide), Yervoy (Ipilimumab), Yescarta (Axicabtagene), Yondelis (Trabectedin), Zaltrap (Ziv-aflibercept), Zanosar (Streptozocin), Zejula (Niraparib), Zelboraf (Vemurafenib), Zevalin (Ibritumomab Tiuxetan), Zoladex (Goserelin), Zolinza (Vorinostat), Zometa (Zoledronic acid) Zortress (Everolimus), Zydelig (Idelalisib), Zykadia (Ceritinib), Zytiga (Abiraterone), or combinations thereof. In some embodiments, the chemotherapeutic drug is selected from: Abraxane (Paclitaxel Albumin-stabilized Nanoparticle Formulation), Afinitor (Everolimus), Erlotinib Hydrochloride, Everolimus, Gemcitabine Hydrochloride, Irinotecan Hydrochloride, Lynparza (Olaparib), Mitomycin, Olaparib, cyclophosphamide, doxorubicin, oxaliplatin, mitoxantrone, Sunitinib Malate, or combinations thereof. In some embodiments, the chemotherapeutic drug is a combination of any of the aforementioned chemotherapeutic drugs.

In some embodiments, a pharmaceutical composition comprising a DNA-PROTAC of this disclosure is administered, e.g., parenterally, at dosage levels of sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the composition described herein is administered at a dose that is below the dose at which the agent causes non-specific effects.

In some embodiments, a pharmaceutical composition comprising a DNA-PROTAC of this disclosure is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Pharmaceutical compositions described herein can be prepared by any method known in the art of pharmacology. In general, such preparatory methods include the steps of bringing the composition comprising a DNA-PROTAC of this disclosure into association with a carrier and/or one or more other accessory ingredients, and then, if necessary and/or desirable, shaping and/or packaging the product into a desired single- or multi-dose unit. The carrier includes the feature that it does not include denaturing agents so as to retain the hybridization complex of the DNA-PROTAC.

### Certain Uses

In certain embodiments, the present invention provides a use of the DNA-PROTACs described herein, or compositions comprising said DNA-PROTACs, for the manufacture of a medicament for inducing a tumor necrosis response in a subject.

In certain embodiments, the present invention provides a use of the composition as described herein for inducing a tumor necrosis response.

In certain embodiments, the present invention provides a use of the composition as described herein for the manufacture of a medicament for treating a disease or disorder in a subject.

### Compositions

Certain embodiments of the invention provide for a composition as described herein for use in medical therapy.

Certain embodiments of the invention provide the use of a composition as described herein for the manufacture of a medicament for inducing an immune response in a subject in combination with at least one therapeutic agent. In some embodiments, the subject is a mammal, which can include or exclude a human.

Certain embodiments of the invention provide a composition as described herein for inducing an immune response, in combination with at least one therapeutic agent.

Certain embodiments of the invention provide the use of a composition as described herein for the manufacture of a medicament for treating a disease or disorder in a subject.

Certain embodiments of the invention provide the use of a composition as described herein for the manufacture of a medicament for treating a disease or disorder in a subject, in combination with at least one therapeutic agent.

Certain embodiments of the invention provide a composition as described herein for the prophylactic or therapeutic treatment a disease or disorder.

Certain embodiments of the invention provide a composition as described herein for the prophylactic or therapeutic treatment of a disease or disorder, in combination with at least one therapeutic agent.

### Formulations

The pharmaceutical combination of the present invention may be formulated with a “carrier.” As used herein, “carrier” includes any solvent, dispersion medium, vehicle, coating, diluent, antibacterial and/or antifungal agent, isotonic agent, absorption delaying agent, buffer, carrier solution, suspension, colloid, and the like. The pharmaceutical combinations can be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; (2) topical application, for example, as a cream, lotion, gel, ointment, or a controlled-release patch or spray applied to the skin; (3) intravaginally or intrarectally, for example, as a pessary, cream, suppository or foam; (4) sublingually; (5) ocularly; or (6) nasally. The carriers of this disclosure do not include a denaturant to retain the hybridization structure of the DNA-PROTAC.

A combination of the present disclosure may be provided in a single formulation. In other embodiments, the pharmaceutical combination of the present disclosure may be provided in separate formulations. A pharmaceutical combination may be formulated in a variety of and/or a plurality of forms adapted to one or more preferred routes of administration. Thus, a pharmaceutical combination can be administered via one or more known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical combination, or a portion thereof, can be administered to a mucosal surface, such as by administration to, for example, the nasal or respiratory mucosa (e.g., by spray or aerosol). A pharmaceutical combination, or a portion thereof, also can be administered via a sustained or delayed release.

A pharmaceutical combination of the present disclosure may be conveniently presented in unit dosage form and may be prepared by methods well known in the art of pharmacy. Methods of preparing a combination with a pharmaceutically acceptable carrier include the step of bringing the pharmaceutical combination of the present disclosure into association with a carrier that constitutes one or more accessory ingredients. In general, a pharmaceutical combination of the present disclosure may be prepared by uniformly and/or intimately bringing the active compound into association with a liquid carrier, a finely divided solid carrier, or both, and then, if necessary, shaping the product into the desired formulations.

In some embodiments, the method can include administering a sufficient amount of the pharmaceutical combination of the present disclosure to provide a dose of, for example, from about 0.1 mg/kg to about 1,000 mg/kg to the subject.

### Administration

As described herein, methods of the invention comprise administering a composition comprising a composition as described herein. In some embodiments, such compositions are formulated as a pharmaceutical composition and administered to a mammalian host, which can include or exclude a human patient in a variety of forms adapted to the chosen route of administration, i.e., orally or parenterally, by intravenous, intramuscular, intraperitoneal or topical or subcutaneous routes.

In some embodiments, the compositions are systemically administered in combination with a pharmaceutically acceptable vehicle. In some embodiments, such compositions and preparations comprise at least 0.1% of active DNA-PROTAC. The percentage of the compositions and preparations may, of course, be varied and may conveniently be between about 0.1 to about 60% of the weight of a given unit dosage form. The amount of active DNA-PROTAC in such therapeutically useful compositions is such that an effective dosage level will be obtained. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. In some embodiments, the liquid carrier or vehicle can be a solvent or liquid dispersion medium comprising a liquid which can include or exclude: water, ethanol, a polyol (for example, glycerol, propylene glycol, liquid polyethylene glycols, and the like), vegetable oils, nontoxic glyceryl esters, and suitable mixtures thereof. The proper fluidity can be maintained by the formation of liposomes, by the maintenance of the required particle size in the case of dispersions or by the use of surfactants.

Sterile injectable solutions are prepared by incorporating the active DNA-PROTAC in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of compounds can be determined by comparing their in vitro activity, and in vivo activity in animal models. Methods for the extrapolation of effective dosages in mice, and other animals, to humans can include U.S. Pat. No. 4,938,949, herein incorporated by reference.

The amount of the DNA-PROTAC required for use in treatment will vary not only with the particular salt selected but also with the route of administration, the nature of the condition being treated and the age and condition of the patient and will be ultimately at the discretion of the attendant physician or clinician.

The DNA-PROTACs of this disclosure may be conveniently formulated in unit dosage form. In one embodiment, the invention provides a composition comprising a compound formulated in such a unit dosage form. The desired dose may conveniently be presented in a single dose or as divided doses administered at appropriate intervals. In some embodiments, the dose interval is selected from two, three, four or more sub-doses per day.

### EXAMPLES

The invention will now be illustrated by the following non-limiting Examples.

### Materials and Methods

Within this application, unless otherwise stated, the techniques utilized may be found in any of several well-known references such as: Molecular Cloning: A Laboratory Manual (Sambrook, et al., 1989, Cold Spring Harbor Laboratory Press), Current Protocols in Molecular Biology (Ausebel et al., Wiley-Interscience, 1988. New York), and PCR Protocols: A Guide to Methods and Applications (Innis, et al. 1990. Academic Press, San Diego, Calif.).

### Small Molecule Synthesis, and Characterizations

General Methods: Unless noted otherwise, all non-aqueous reactions were conducted in oven-dried glassware under argon or nitrogen atmosphere. Reagents were commercially available and used without further purification, while anhydrous solvents were procured as the highest grade from Sigma-Aldrich. Thin-layer chromatography using 0.25 mm Silicycle silica gel 60 F254 plates was employed for reaction monitoring. Flash column chromatography utilized for small molecule purification using commercial 40-60 mesh silica gel column. Yields are presented as isolated yields of spectroscopically (NMR&LC/MS) pure compounds. ¹H and ¹³C NMR spectra were acquired using 400 and 500 MHz Varian spectrometers. Chemical shifts are expressed in parts per million (ppm, δ) referenced to the residual ¹H resonance of the solvent (CDCl₃, 7.26 ppm, and DMSO-d₆, 2.49 ppm). 13C spectra are referenced to the residual 13C resonance of the solvent (CDCl₃, 77.16 ppm, and DMSO-d₆, 39.52 ppm). Splitting patterns are denoted as follows: s, singlet; br, broad; d, doublet; dd, doublet of doublets; t, triplet; q, quartet; m, multiplet.

### General Biology Methods

### Cell Culture

U-251 MG Glioblastoma Cell Line was grown in Dulbecco's Modified Eagles Medium (DMEM) containing 10% heat inactivated fetal bovine serum (FBS), streptomycin (5 μg/mL) and 5 U/mL penicillin 95 U/mL). All cell lines were maintained, and cell culture experiments were carried out in humidified incubators at 37° C. and 5% CO₂ supplementation.

### DNA-PROTAC Transfection

One day prior to DNA-PROTACs transfection, cells were propagated into 6 cm cell culture dishes containing appropriate complete growth medium. Prior to transfection, complete medium was replaced with transfection medium (5% FBS, no Penstrep). DNA-PROTACs transfection was performed using lipofectamine 3000 reagent according to the protocols provided by the manufacture. All transfections were carried out in 6-cm dishes with 3 mL of media and concentrations of DNA-PROTACs were calculated according to this volume (3 mL). Briefly, for 50 nM concentration, 5 μL from a 30 UM DNA-PROTACs stock and 12 μL p3000 reagent was added to a tube containing 125 μL of OPTIM-MEM and 8 μL of Lipofectamine 3000 reagent was added to a separate tube containing 125 μL of OPTIM-MEM. Two tubes were incubated for 5 minutes at room temperature and DNA-PROTACs containing OPTI-MEM was then slowly added to the second tube with lipo3000. The solution in the tube was mixed well by pipetting up and down several times. After incubating for 15-20 minutes at room temperature, 250 μL of DNA-PROTACs-lipofectamine complex was added drop wise onto cells containing the transfection medium. Transfection medium was mixed well before transferring the plate into the incubator. After appropriate time, cells were either harvested or replaced with fresh medium and incubated for desired time point prior to harvesting. For proteasome inhibition assay, 5 μM of MG-132 was incubated with cells for 2 h prior to transfection of DNA-PROTACs. Then, cells were incubated for another 12 h before lysing cells. Cell lysates were prepared by incubating cells in RIPA lysis buffer (25 mM Tris pH 7.6, 150 mM NaCl, 1% NP40, 1% deoxycholate, 0.1% SDS, 1× protease inhibitor cocktail from Roche and 1 mM of PMSF) on ice for 30 minutes and cell lysate was clarified by centrifugation at high speed (15 000 rpm) for 15 minutes. Clear supernatant was collected for further experiments.

### Annealing Reaction

RP-HPLC purified single stranded oligo conjugated to CRBN ligand (DNA-E3i) and its reverse complement oligo conjugated with CDK6 ligand (cDNA-CDK6i) were dissolved in ultra-pure water. Single stranded DNA-E3i and single stranded reverse complement oligos were mixed 1:1 molar ratio (final concentrations of DNA-PROTACs were set to 30 μM) in 1× annealing buffer (10 mM Tris, pH 7.5, 50 mM NaCl and 1 mM EDTA) and incubated for 5 minutes in a water bath at 95 degrees Celsius, then cool down to 4 degrees Celsius. Double stranded DNA-PROTACs were mixed by gently vertexing and aliquoted and stored at −20° C.

### Western Blotting

Protein concentration in all the cell lysates were measured by BCA protein assay kit and equal amounts from each lysate were mixed with 4×loading dye and boiled for 5 minutes followed by 2 minutes centrifugation prior to loading into SDS-PAGE gel. Next proteins on the SDS-PAGE gel were transferred to a Nitrocellulose membrane by western blotting and the membrane was blocked with 5% milk in TBST (0.05% Tween 20) for 1 h. Primary antibodies (all Abcam antibodies were diluted 1:5000) were prepared in TBST with 2.5% milk and membranes were incubated overnight at 4° C. On the following day, membrane was washes for 15 minutes (Incubate for three times, 5 minutes each) and appropriate secondary antibodies (1:5000) were prepared in TBST and incubated with the membrane for 1 h at room temperature (RT). Membrane was washed for 15 minutes with TBST (incubate for three times, 10 minutes each) prior to imaging.

### Immunofluorescence Staining

U-251 MG cells were seeded into 10 mm confocal dishes (Ibidi) and incubated overnight. Subsequently, cells were quickly washed thrice with ice-cold PBS and then fixed with 4% paraformaldehyde for 20 min. After fixation, cells were washed with PBS and permeabilized with 0.1% Triton X-100 at room temperature for 10 min. The samples were then incubated in a blocking solution for 1 h at room temperature, followed by incubation with primary antibody overnight at 4° C. The primary antibody used was mouse CDK6 antibody (Abacm, 1:100). After washing thrice with PBST (PBS with 0.1% Tween 20), the samples were incubated with the secondary antibody in the dark for 1.5 h at room temperature. Cells were washed 3 times in PBST for 5 minutes each then incubated in the appropriate secondary antibodies supplemented CoraLite488-conjugated Goat Anti-Rabbit IgG(H+L) (Proteintech, 1:100) for 3 hrs. The nucleus was counterstained with DAPI (Hoechst 3342, 1 ug/mL) for 20 mins followed by 3 times wash in PBST. Finally, the slides were Imaging by Nikon X confocal.

### Mouse Model Methods

In certain embodiments, the methods of this disclosure can treat cancer in a relevant model. Mouse models for pancreatic cancer to which the methods of this disclosure are expected to demonstrate the treatment of cancer are described in Herreros-Villanueva et al., Mouse models of pancreatic cancer World J Gastroenterol. (2012) Mar. 28; 18(12): 1286-1294. doi: 10.3748/wjg.v18.i12.1286, PubMed ID: 22493542), incorporated herein by reference.

### Example 1

### Synthesis of Representative Embodiments of DNA-Based Chimeras (DNA-PROTAC) for Targeted Protein Degradation of CDK6

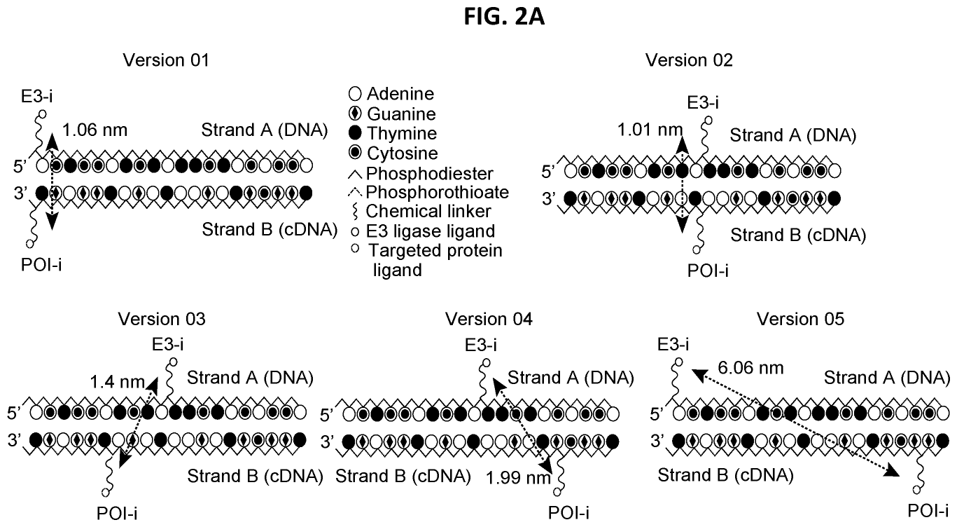

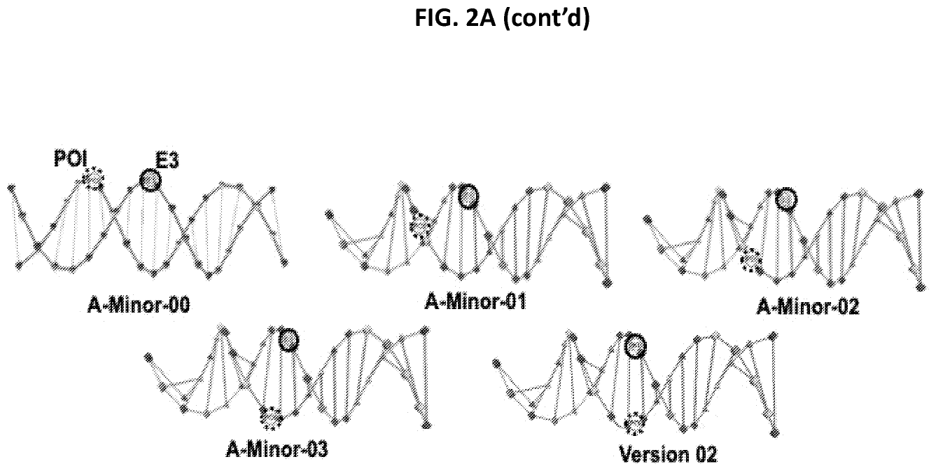

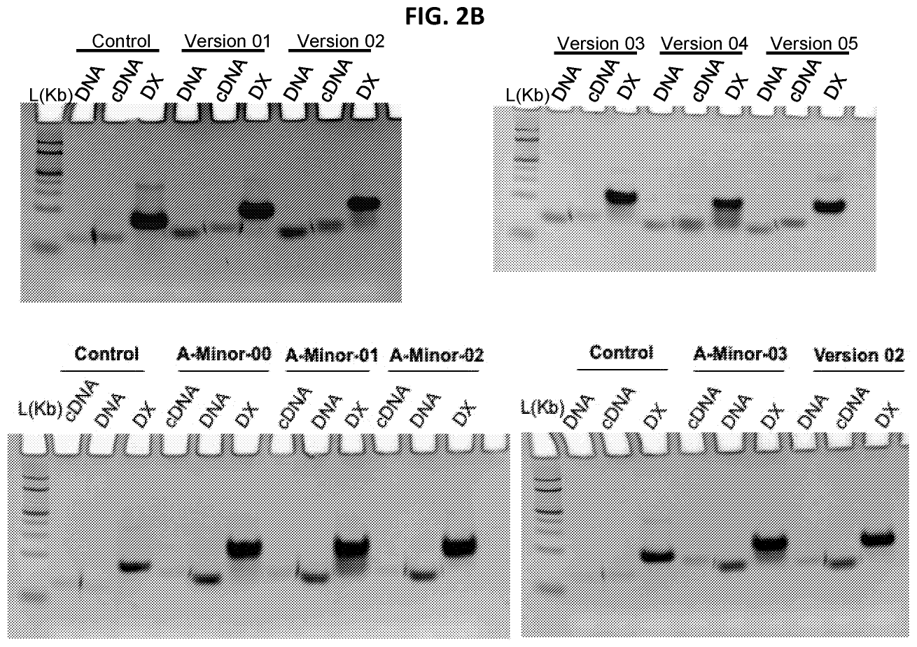

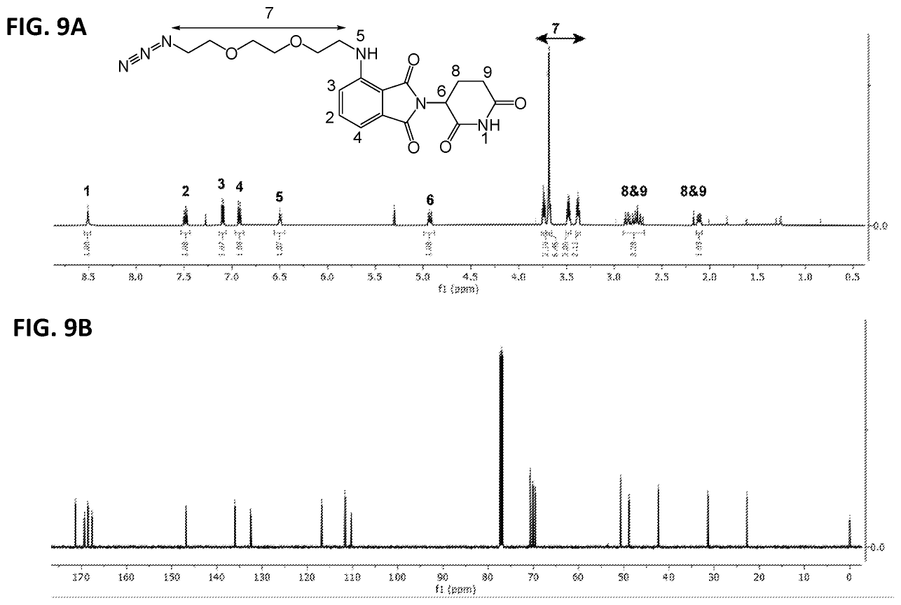

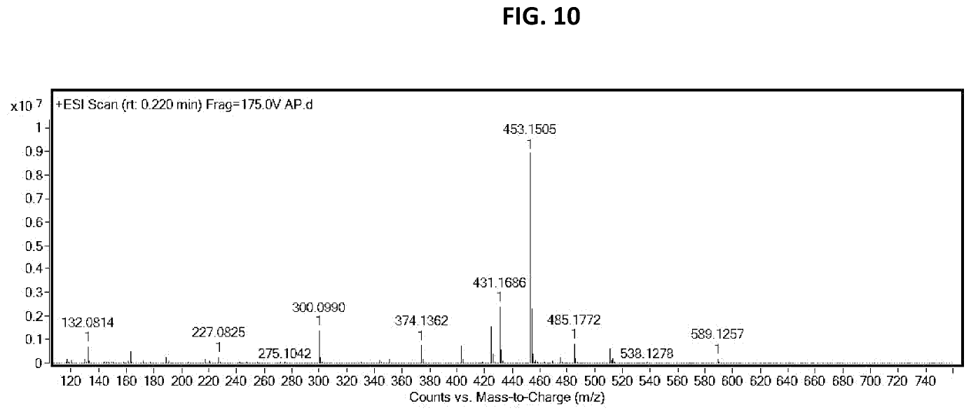

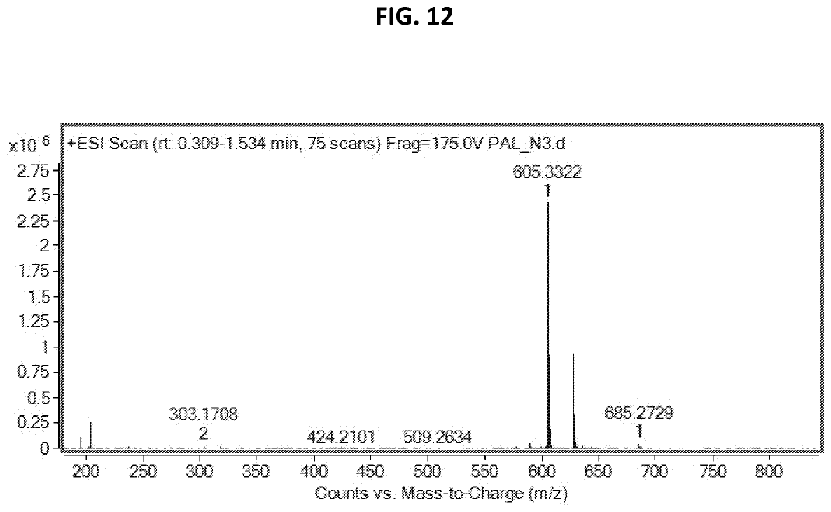

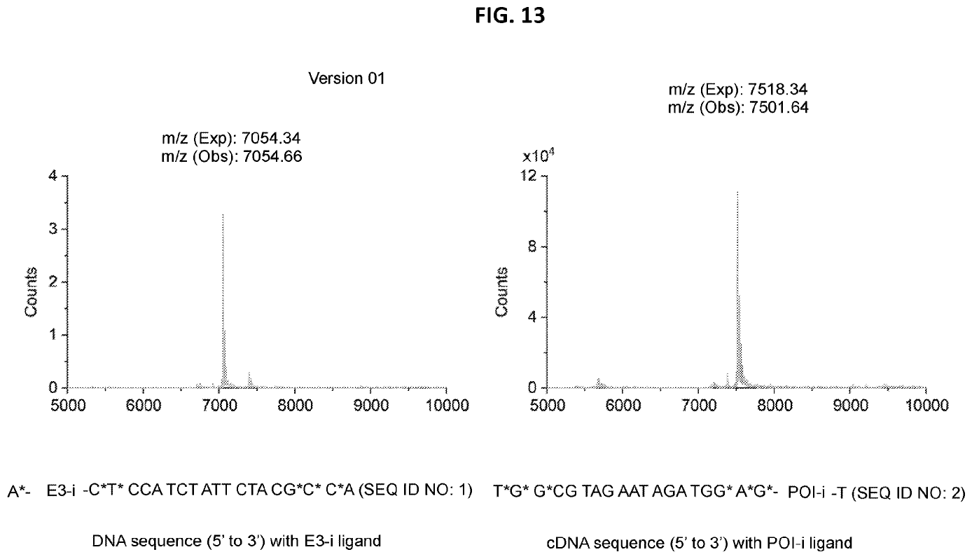

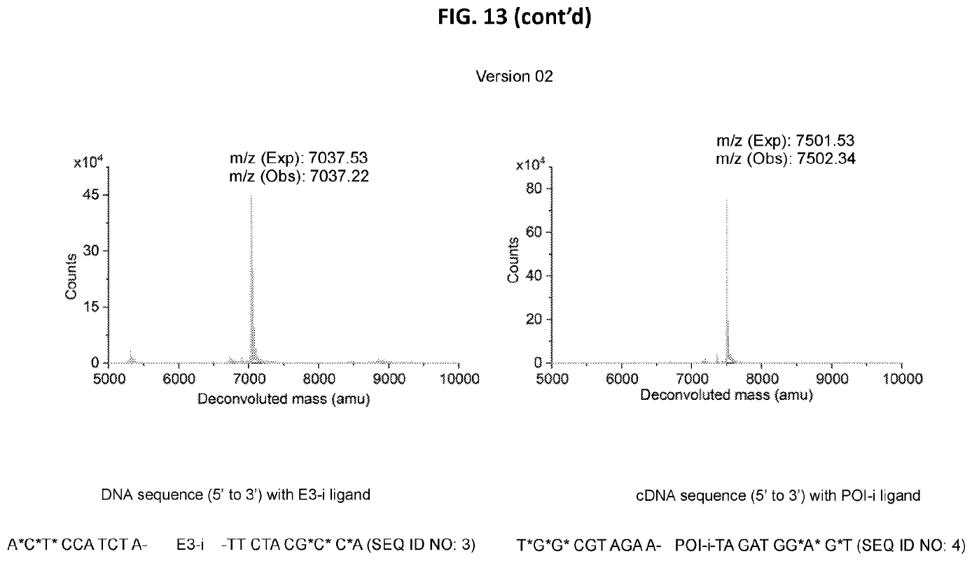

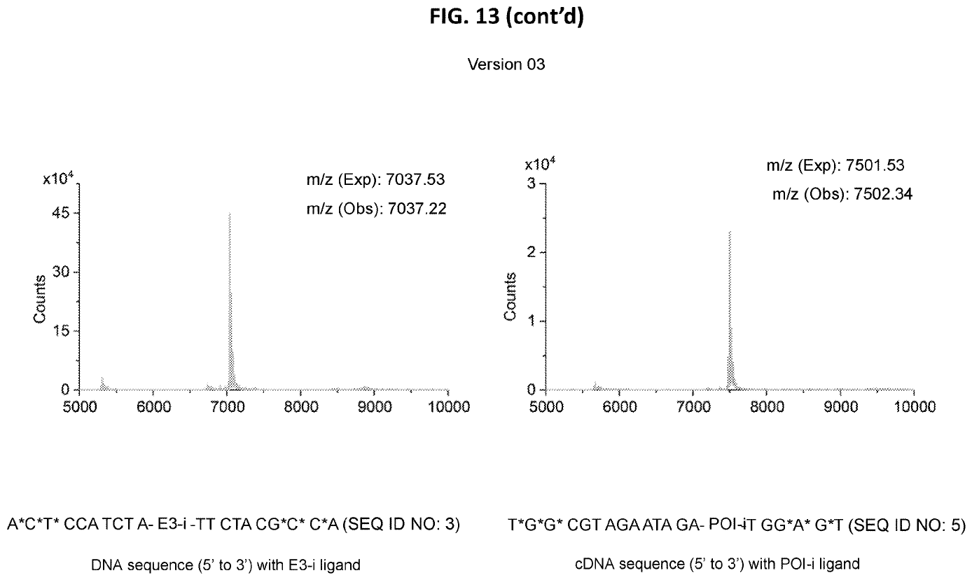

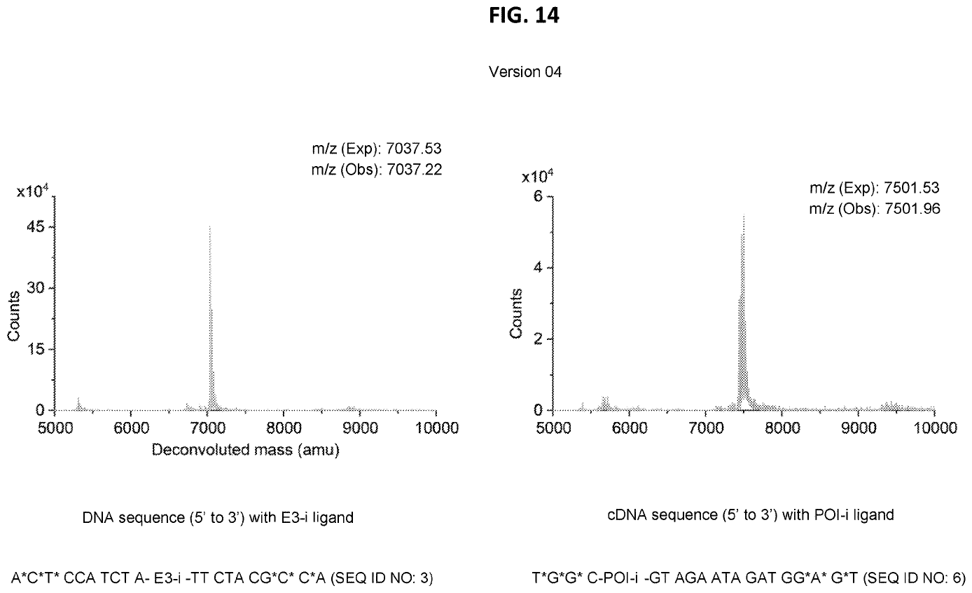

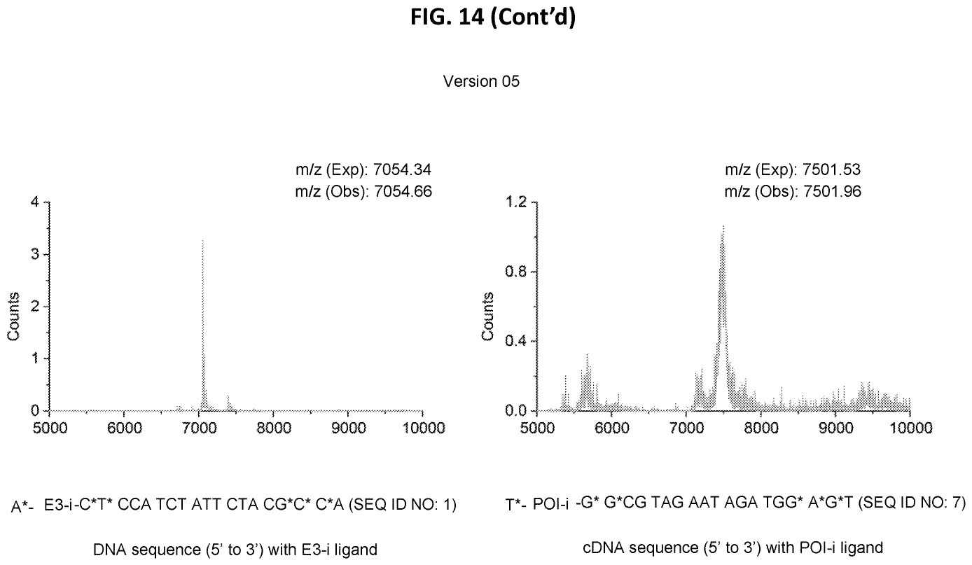

Optimization of spatial organization-activity relationship of DNA-PROTAC library: The success of small molecule based PROTAC largely depends on the optimal linker length. (Cyrus, K.; Wehenkel, M.; Choi, E. Y.; Han, H. J.; Lee, H.; Swanson, H.; Kim, K. B. Impact of Linker Length on the Activity of PROTACs. Mol Biosyst 2011, 7 (2), doi.org/10.1039/c0mb00074d; Chen, Y.; Tandon, I.; Heelan, W.; Wang, Y.; Tang, W.; Hu, Q. Proteolysis-Targeting Chimera (PROTAC) Delivery System: Advancing Protein Degraders towards Clinical Translation. Chemical Society Reviews. Royal Society of Chemistry Jun. 17, 2022, pp 5330-5350. doi.org/10.1039/d1cs00762a; Poongavanam, V.; Atilaw, Y.; Siegel, S.; Giese, A.; Lehmann, L.; Meibom, D.; Erdelyi, M.; Kihlberg, J. Linker-Dependent Folding Rationalizes PROTAC Cell Permeability. J Med Chem 2022, 65 (19). doi.org/10.1021/acs.jmedchem.2c00877)). However, it remains challenging to control the spatial orientation using flexible chemical likers. The orientational control of ligands can influence protein facing with E3 ligase complex that may facilitate ubiquitination as well as selective ubiquitination of single protein in case of protein complexes (e.g., CDK6/CDK4) (Lebraud, H.; Wright, D. J.; Johnson, C. N.; Heightman, T. D. Protein Degradation by In-Cell Self-Assembly of Proteolysis Targeting Chimeras. ACS Cent Sci 2016, 2 (12). doi.org/10.1021/acscentsci.6b00280; Li, B.; Ran, T.; Chen, H. 3D Based Generative PROTAC Linker Design with Reinforcement Learning.) Therefore, in this example we are interested in exploring a small library of DNA-PROTAC constructs synthesized with spatial control on distance and orientation between two small molecules. The key hypothesis of our work is to position small molecules onto a DNA scaffold platform with a pre-defined spatial organization and orientational control. As a starting test module, we utilized a small DNA duplex (20 bp; ~6.8 nm) as a scaffold that was functionalized with small molecules via a general procedure: The conjugation of small molecule ligands pre-modified with polyethylene glycol (PEG) azide to the synthesized single-strand (ssDNA) via Strain-promoted azide-alkyne click (SPAAC) chemistry. The small molecules used for this work are Palbociclib (targeting ligand for E3 ligase) and Pomalidomide (targeting ligand for CDK4/CDK6). The organization (distance-control) and orientation (angle-control) of small molecule ligands in DNA-PROTAC is achieved by manipulating the position of internal amino-serinol phosphoramidite during DNA synthesis. This position provides the attachment site for DBCO-NHS Ester, which undergoes SPAAC with the azide handle on the targeting ligands on either ssDNAs of the DNA-PROTAC duplex. Our data demonstrated that this conjugation reaction occurs with a yield of over 98%, regardless of whether it takes place at the internal or terminal position of the DNA scaffold (FIG. 2B). Consequently, we utilized this strategy to prepare various DNA-PROTAC constructs for studying: 1). Distance-effect: to investigate with an increment in the average distance (nm) between POI-i (0, 7) and E3-i (1-6) ligands, and 2). Angular-effect: to explore the effect of varying orientation from 0° (parallel) to 180° (perpendicular) between the two ligands with a 36° increment for each base turn (FIG. 2A). To investigate distance effect, five different versions were synthesized using following general protocol (FIG. 2A). The conjugation of small molecule ligands pre-modified with PEG azide to the synthesized ssDNA via SPAAC. The small molecules used for this work are Palbociclib (targeting ligand for E3 ligase; E3-i) and Pomalidomide (targeting ligand for CDK4/CDK6; POI-i), which were tested thoroughly by us during the preliminary studies. The distance-control of small molecule ligands in different version of DNA-PROTAC (FIG. 2A) was achieved by manipulating the position of internal amino-serinol phosphoramidite during solid-phase DNA synthesis. This position provides the attachment site for DBCO-NHS Ester, which undergoes SPAAC with the azide handle on the targeting ligands on either ssDNAs of the DNA-PROTAC duplex. Each ssDNA strand obtained was purified using (reverse phase high performance liquid chromatography) RP-HPLC, lyophilized, redissolved in Milli-Q water, and characterized by native polyacrylamide gel electrophoresis (PAGE) (FIG. 2B) and Quadrupole Time of Flight Liquid Chromatography Mass Spectrometry (QTOF LC/MS) (FIG. 9-14). The above constructs are chosen to perform a systematic screening of the position of two targeting ligands (distance-control) along the major and minor grooves, on opposite and similar ends of the DNA scaffold, covering a distance ranging from 9.9 Å to 64.2 Å. Additionally, in future comparing with angular effect will allow us to determine the most crucial factor responsible for protein degradation.

### Example 2

### Evaluate the Distance Effect of DNA-PROTACs on Reducing the CDK6 Protein Level.

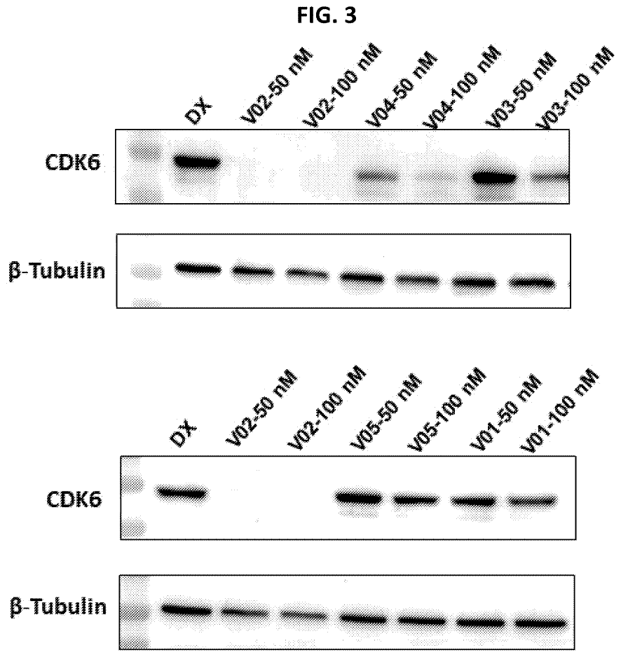

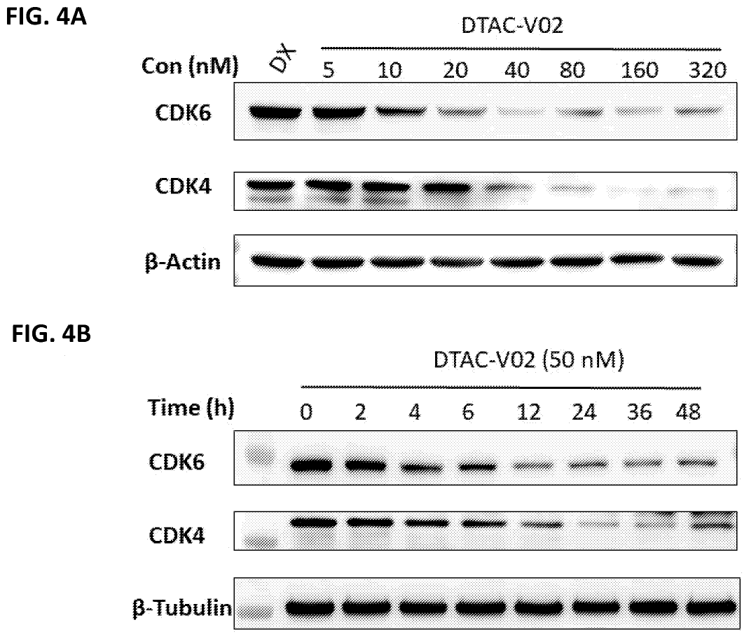

The degradation efficiency of various DNA-PROTACs was evaluated through their transfection into U251 cells, conducted simultaneously to ensure uniform experimental conditions. A double-stranded DNA sequence without any ligand was utilized as a negative control, providing a baseline for comparison. Following a 14-hour treatment period, a duration selected based on preliminary time-course studies, the cells were harvested using trypsin digestion. The lysates obtained were then subjected to SDS-PAGE, followed by Western blot analysis to assess protein degradation. As depicted in FIG. 3-4, DNA-PROTAC Version-02 (V02) exhibited the most pronounced degradation of CDK6. This was evidenced by the significantly reduced visibility of CDK6 bands on the gel, in stark contrast to the control, at both 50 nM and 100 nM concentrations. This finding highlights the efficacy of DNA-PROTAC V02 in targeting and reducing CDK6 protein levels. In comparison, DNA-PROTAC Versions 03 and 04 also facilitated protein degradation, albeit with varying degrees of efficiency. Version 03 was observed to substantially reduce CDK6 protein levels at 100 nM. However, it did not match the effectiveness of V02. Version 04, on the other hand, showed a more pronounced reduction in protein levels at a lower concentration of 50 nM. Interestingly, Version 05, characterized by the largest ligand distance of approximately 60.2 Å, did not exhibit any discernible protein degradation at either concentration. Similarly, Version 06, incorporating both an E3 ligand and a POI ligand (a CDK6 inhibitor) at the terminal ends of the DNA duplex, also failed to show significant protein degradation. These observations suggest that the spatial arrangement, specifically the distance and angle between the E3 ligand and POI ligand, can be modulated to influence the protein degradation efficiency of DNA-PROTACs. Based on these results, the structural configuration of DNA-PROTACs, particularly the spatial relationship between their constituent ligands, can be modulated to mediate CDK6 protein degradation.

Similar results are expected with other cell lines to model other cancer types to demonstrate the methods described herein can be used to treat a broad class of cancers. For example, human Ewing's sarcoma cell line A-673, and a human leukemia cell line THP-1 and HL-60 can also be used using similar methods as described herein, to demonstrate that the DNA-PROTACs can be used to kill a cancer cell, and therefore treat a subject having cancer.

### Example 3

### Evaluation of the Angular Effect of DNA-PROTACs on Reducing the CDK6 Protein Level

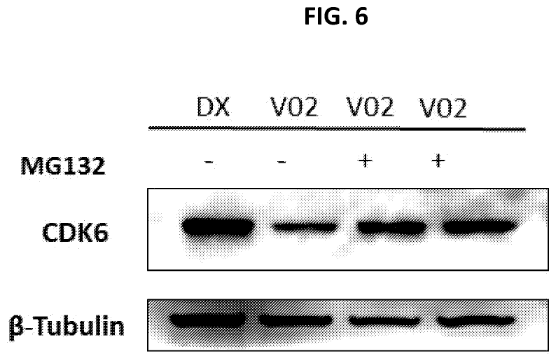

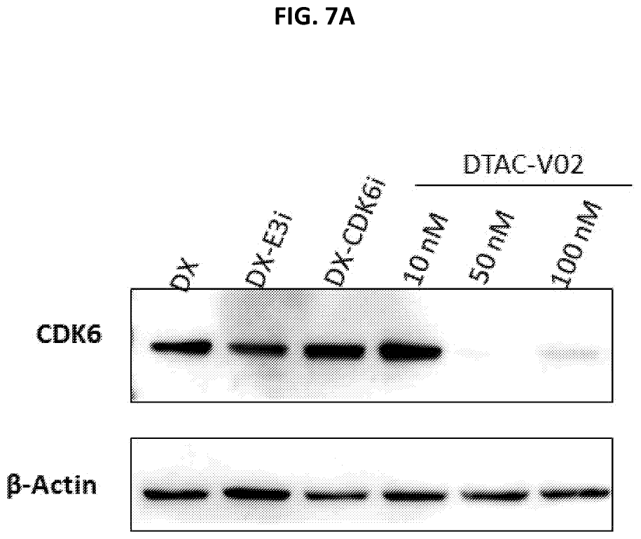

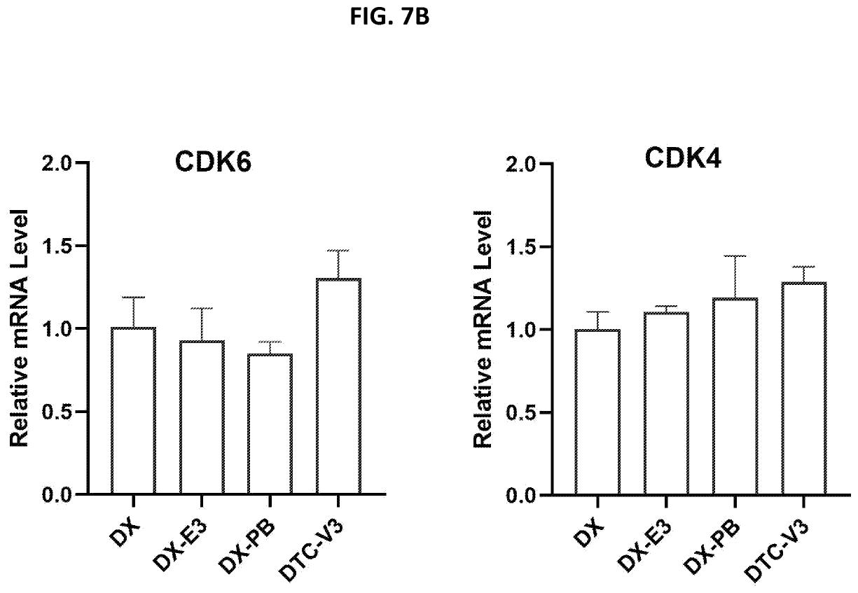

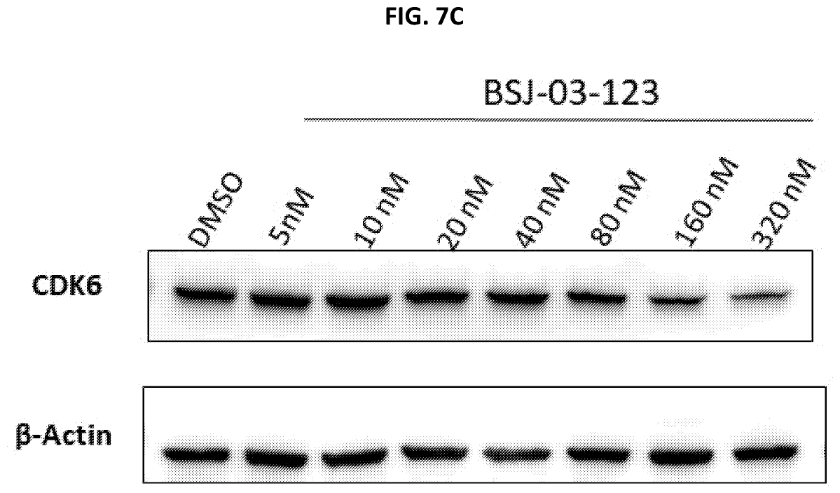

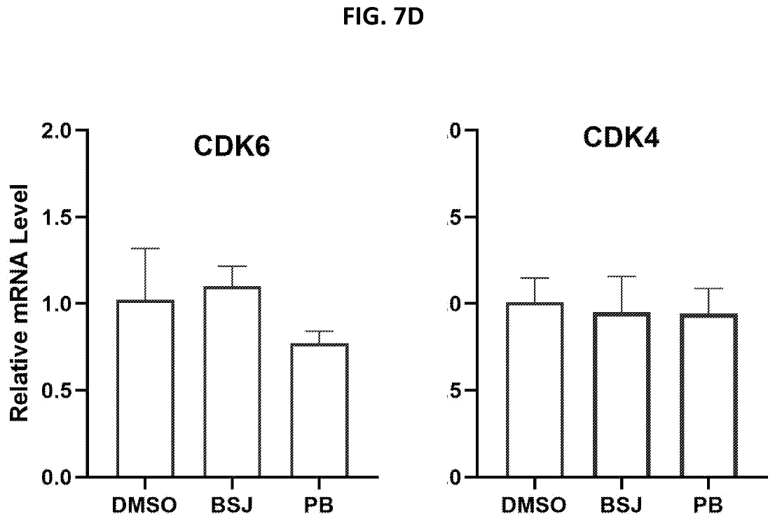

To investigate the influence of ligand orientation on protein degradation efficiency, a series of DNA-PROTACs named A-minor-00, A-minor-01, A-minor-02, and A-minor-03, along with a variant DNA-PROTAC-VO2, were engineered. These compounds spanned an orientation range from 0° (parallel) to 180° (perpendicular) between the E3 ligand and the protein of interest (POI) ligand. Their ability to form DNA duplexes was confirmed through native gel electrophoresis, as depicted in FIG. 6. Subsequently, U251 cells were treated with these angular-dependent DNA-PROTACs, using double-stranded DNA as a negative control. A treatment duration of 14 hours, determined from initial time-course experiments, was employed before cell harvest via trypsin digestion. The resultant cell lysates were analyzed by SDS-PAGE and Western blot to evaluate protein degradation. As illustrated in FIG. 7, A-minor-00 demonstrated marginal CDK6 degradation. In contrast, A-minor-01 and A-minor-02 facilitated substantial degradation at a concentration of 100 nM, while A-minor-03 and DNA-PROTAC-VO2 achieved significant CDK6 degradation beginning at 20 nM. These findings indicate that the efficiency of protein degradation is modulated by the angular disposition between the E3 and POI ligands.

### Example 4

DNA-PROTAC-V02 Efficiently Induced CDK4 and CDK6 Degradation with Dose- and Time Dependent Manner.

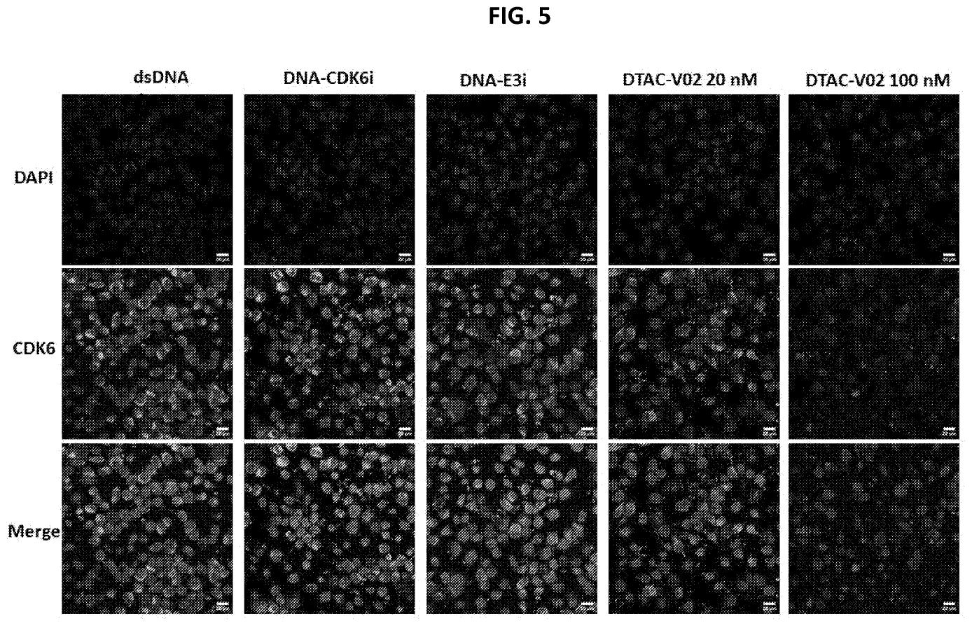

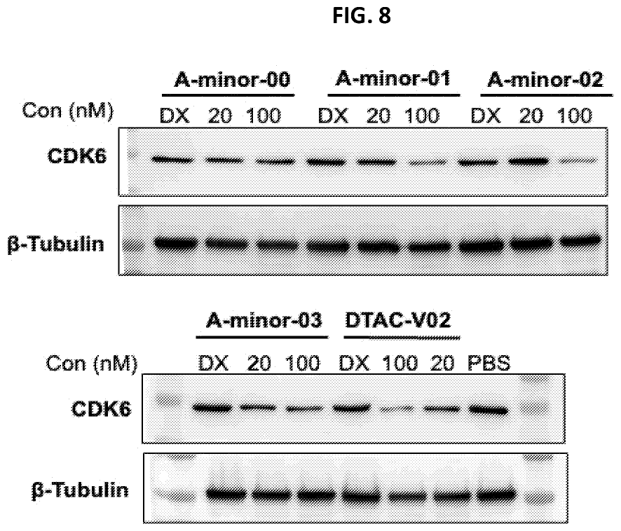

In light of the remarkable degradation efficiency observed for CDK6 with DNA-PROTAC-V02, representative DNA-PROTACs were created to measure their impact on CDK4 protein, given the inhibitor for CDK6 shares binding affinity with CDK4. As depicted in FIG. 6-8, DNA-PROTAC-V02 initiated a dose-dependent degradation of CDK6, commencing at a remarkably low concentration of 10 nM, and exhibiting an intensified effect with increasing concentrations. Noteworthy is the simultaneous reduction in CDK4 protein levels, initiated at 40 nM, underscoring DNA-PROTAC-V02's capacity for dual protein degradation. In conclusion, DNA-PROTAC-V02 demonstrated the ability to induce dual degradation of CDK4 and CDK6 proteins in a dose-dependent manner. To further unravel the functional characteristics of DNA-PROTAC-V02, we conducted immunofluorescence staining under various treatment conditions, including control conditions (dsDNA only, dsDNA with CDK6 inhibitor, dsDNA with E3 ligand), as well as with DNA-PROTAC-V02 at concentrations of 20 nM and 100 nM. As shown in FIG. 5, Immunofluorescence imaging revealed a weakened intracellular green fluorescence induced by DNA-PROTAC-V02 compared to controls (dsDNA, dsDNA-CDK6-i, dsDNA-E3-i). This observation provided additional evidence of DNA-PROTAC-V02's efficacy in mediating dual protein degradation, particularly in reducing CDK6 protein levels. Furthermore, our investigation revealed that DNA-PROTAC-V02 induced dual degradation of CDK4 and CDK6 proteins in a time-dependent manner. As illustrated in FIG. 4, the degradation of CDK4/6 proteins commenced after a 48-hour treatment and sustained degradation for up to 48 hours. These findings offer valuable insights into the multifaceted degradation capabilities of DNA-PROTAC-V02, showcasing its utility for targeted modulation of both CDK6 and CDK4 proteins.

### Example 5

### Demonstrate the Mechanism of DNA-PROTAC-V02-Mediated Protein Degradation

To comprehensively understand the mechanisms governing protein degradation induced by DNA-PROTAC-V02, a chemical linker denoted as BSJ-03-123, was employed which targeted CDK6, in U251 cells. The ensuing results, depicted in FIG. 7A-D, reveal a discernible reduction in protein levels under 160 nM treatment. Despite this, the exceptional efficacy of DNA-PROTAC-V02 becomes apparent, achieving over 80% degradation at a minimal concentration of 50 nM (FIG. 7A). To ascertain the specificity of DNA-PROTAC-V02's protein degradation mechanism and its potential impact on mRNA levels, we subjected U251 cells to treatments with DX, DX-E3i, DX-CDK6i, and DNA-PROTAC-V02 (100 nM, 6 hours). Subsequent RNA isolation and RT-PCR detection enabled a quantitative assessment of CDK6 and CDK4 mRNA expression levels. FIG. 7B, D unequivocally demonstrates that DNA-PROTAC-V02 treatment does not elicit a decrease in mRNA levels for CDK6 and CDK4—a finding consistent with observations in the chemical PROTAC treatment group. This underscores DNA-PROTAC-V02's capacity to selectively induce protein degradation without affecting mRNA expression. To further corroborate the mechanistic intricacies of DNA-PROTAC-V02, we probed its interaction with the proteasome pathway. Prior to transfection with DNA-PROTAC-V02 (50 nM), U251 cells underwent pre-treatment with the proteasome pathway inhibitor (MG132) for 1 hour to temporally impede proteasome function. Following an 8-hour treatment period, cells were collected, and Western blot analysis was conducted. A reduction in CDK6 protein without MG132 treatment was observed, while MG132 intervention led to the rescue of CDK6 proteins in both experimental trials. In conclusion, DNA-PROTAC-V02, a representative embodiment of this disclosure, operates by selectively leveraging the proteasome pathway, providing a nuanced understanding of its protein degradation mechanism.

### Example 6

### Multi-Targeting DNA-PROTACs

In complex diseases such as cancer, a prevalent limitation of single-target drugs lies in their susceptibility to rapid development of drug resistance, leading to further morbidity and tumor relapse. DNA scaffolds can be used for the functionalization of multi-targeting DNA-PROTAC ligands-targeting multiple proteins as well as targeting multiple domains in a single protein. Utilizing the hierarchical self-assembly properties of DNA scaffolds from basic monomeric units, advantages in synthetic complexity can be realized over conventional small molecules as target ligands. Distinct arms can strategically position DNA-PROTAC targeting ligands, cyto-directing agents, and cell-targeting moieties. To assess multitargeting capabilities, DNA-PROTACs can be made for the concurrent targeting of different CDK protein family members, specifically CDK6, CDK9 (THAL-SNS-032, or B03), and CDK2 (AZD5438, or AT7519-7). Small-molecule inhibitors designed for CDK protein targeting is modified with an azide handle, purified, and subsequently linked with DNA strands containing a DBCO group. DNA nanostructures can be thermally annealed, employing constituent functionalized single-stranded DNA strands, followed by purification using established techniques, including PAGE gel electrophoresis, spin filtration, and spin gradient methods. Another aspect of positioning multiple single protein targeting ligands within a DNA nanostructure is to overcome the hook effect. This approach can be useful when using high concentration of DNA-PROTAC and to counter formation of E3 ligase:DNA-PROTAC (1:1) and POI:DNA-PROTAC (1:1) complexes, also known as the “hook effect.”

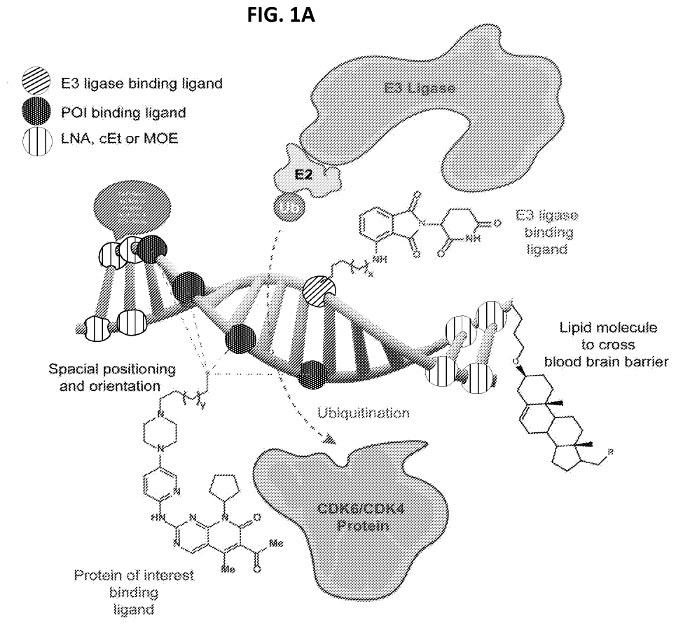

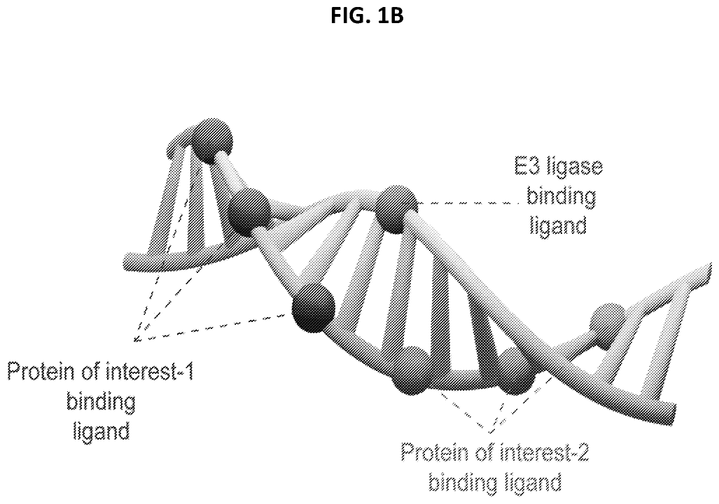

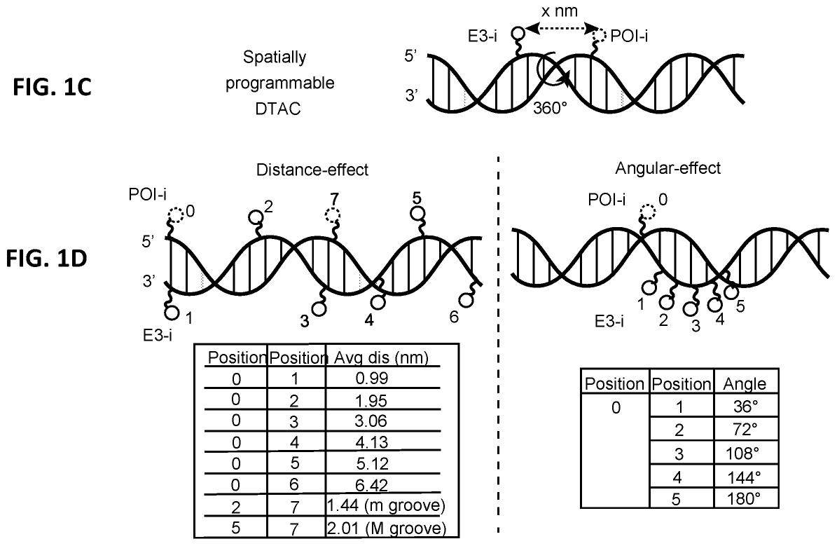

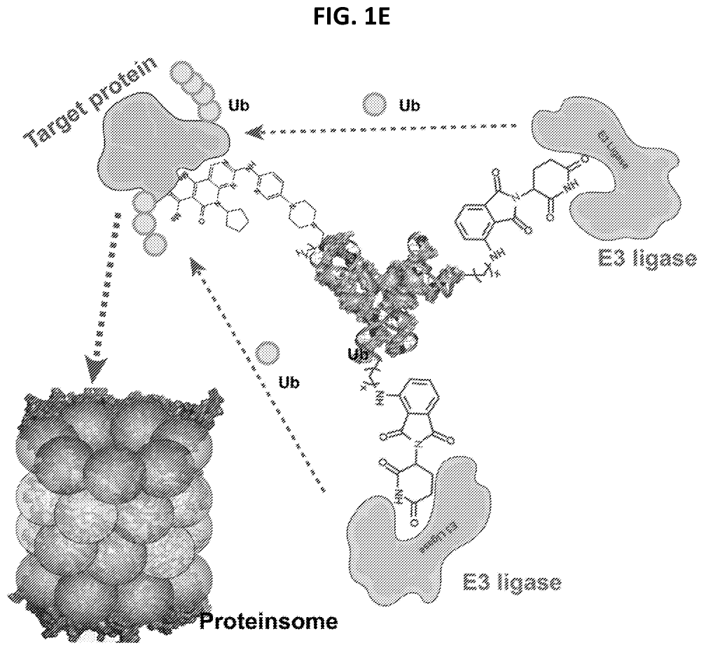

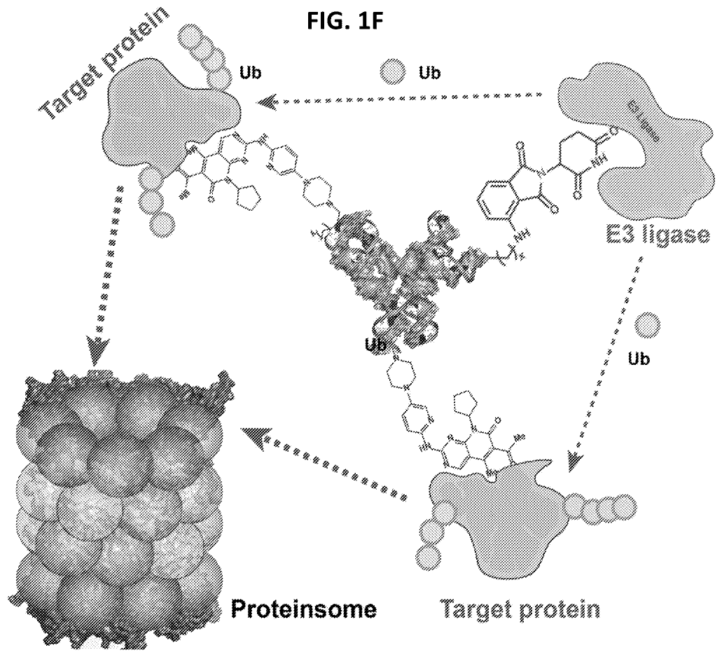

DNA-PROTACs can be developed based on similar principles as described herein to modify different ligands and thus degrade different target proteins. Here, two basic design of DNA-PROTACs can be created, one is recruiting multiple E3 ligase to degrade target proteins as shown in FIG. 1E. Based on similar DNA design, multitargets degradation also could be achieved by changing the DNA sequence to conjugate with the same or different protein ligands (FIG. 1F). More importantly, due to the high programmability and biocompatibility of DNA nanostructures, the design of degradation machines with different modules will be possible. To validate the universal application of DPCs to different targets, we will synthesis the new DPCs of some well-known oncogenes such as bromodomain-containing protein 9 (BRD9), epidermal growth factor receptor (EGFR), BTK and those listed in Table 1 and evaluate the degradation efficacy by the methods described herein.

### Example 7

### Development of DNA Scaffold-Based Targeted Protein Degradation Platform (DNA-PROTAC).

### Spatial Organization-Activity Relationship of a DNA-PROTAC Library.

The success of small molecule based PROTAC largely depends on the optimal linker length. However, it remains challenging to control the spatial orientation when using flexible chemical likers. The orientational control of ligands can influence protein facing with E3 ligase complex that may facilitate ubiquitination, as well as selective ubiquitination of a single protein in the case of a protein complex. The inventors have developed a small library of DNA-PROTAC constructs synthesized with spatial control of distance and orientation between two small molecules to position small molecules onto a DNA scaffold platform with exquisite control of pre-defined spatial arrangement and orientations. A small DNA duplex (20 bp; ~6.8 nm) as a scaffold can be functionalized with small molecules via a two-step procedure: 1) insertion of an amino-modifier phosphoramidite into ssDNA during solid-phase oligonucleotide synthesis; and 2) conjugation of small molecule ligands pre-modified with PEG azide to the synthesized ssDNA via SPAAC. The small molecules used in this example are pomalidomide (targeting ligand for E3 ligase) and palbociclib (targeting ligand for CDK4/CDK6). The arrangement (distance control) and orientation (angle control) of small molecule ligands in DNA-PROTAC is achieved by manipulating the position of internal amino-serinol phosphoramidite during DNA synthesis. This position provides the attachment site for DBCO-NHS Ester, which undergoes SPAAC with the azide handle on the targeting ligands on either ssDNAs of the DNA-PROTAC duplex. This conjugation reaction occurs with a yield of over 98%, regardless of whether it takes place at the internal or terminal position of the DNA scaffold. This strategy demonstrates the preparation of selected DNA-PROTAC constructs for studying: 1) Distance control: to investigate with an increment in the average distance (nm) between POI-i (0, 7) and E3-i (1-6) ligands, and 2) Angle control: to explore the effect of varying orientation from 0° (parallel) to 180° (perpendicular) between the two ligands with a 36° increment for each base turn. Following conjugation, each ssDNA strand obtained is purified using RP-HPLC, lyophilized, redissolved in Milli-Q water, and characterized by QTOF LC/MS. The DNA-PROTACs are selected to perform a systematic screening of the position of two targeting ligands (distance control) along the major and minor grooves, on opposite and similar ends of the DNA scaffold, covering a distance ranging from 9.9 Å to 64.2 Å.

### Example 8

### Demonstration of DNA-PROTAC Target Selectivity.

U251 cells treated with DNA-PROTAC displayed a significant and dose-dependent reduction in both CDK4 and CDK6 protein levels. In contrast, treatment with the DNA duplex alone, DNA-E3 ligand, or DNA-CDK6 ligand did not induce notable protein degradation. Considering that CDK4/6 forms a protein complex with cyclin D1, cyclin D1 protein may also undergo degradation via proximity-induced mechanisms. Western blot screening assays can be used to examine the responses of several other CDKs and kinases to DNA-PROTAC, including Cyclin D3, CDK1, CDK2, CDK4, CDK5, and CDK9, to DNA-PROTAC treatment. Furthermore, to identify additional proteins that may be subject to degradation by DNA-PROTAC due to potential unspecific binding and regulatory pathways. To further characterize the formation of functional ternary complexes in which DNA-PROTAC binds with CDK4/6 proteins and E3 ligases simultaneously, Surface Plasmon Resonance (SPR) assays can be used to to assess the binding affinities of different DNA-PROTAC variants to their respective target proteins. Since the degradation process relies on the ubiquitinproteasome pathway, proteasome inhibitors which include MG132 can be used to rescue target degradation. Additionally, ligand competitive assays is employed to compete with DNA-PROTAC binding to either targets or E3 ligases, thereby rescuing degradation and confirming the formation of functional ternary complexes within cells.

### Example 9

### Creating Representative DNA-PROTACs for Undruggable Targets.

Not all proteins possess conventional active sites suitable for binding with small molecule-based drugs, often referred to as “undruggable” protein targets. The DNA-PROTACs of this disclosure involve small molecule ligands known to interact with specific protein targets. To overcome the limitations posed by the lack of suitable ligands for certain proteins, in some embodiments, alternative binding agents as targeting ligands, including macrocyclic peptides, stapled peptides, and monobody proteins. These novel binders can target proteins when traditional small molecules are not viable options. In some embodiments, the alternative binding agents can include orp exclude: a cyclic peptide that showed preferential binding with GTP bound KRAS (G12D) protein, or a monobody (12VC1) that targets active state of KRAS (G12V) and KRAS (G12C) mutants. Conjugation of these alternative binding agents to DNA can be accomplished through two distinct approaches. First, cysteine-modified alternative binding agents can be modified with hetero-bifunctional small molecule-based linkers, such as succinimidyl 4-(N-maleimidomethyl)cyclohexane-1-carboxylate (SMCC). Second, for alternative binding agents containing an intrinsic cysteine functional group, conjugation via click chemistry can be used to introduce functional groups like azide or alkynes by non-canonical amino acids. Peptide synthesis can be conducted using a peptide synthesizer, while minibinder proteins can be recombinantly expressed in E. coli. Following conjugation, purification can be carried out through PAGE, FPLC, or ion exchange chromatography, with thorough characterization using mass spectrometry. As a represeantive embodiment of an undruggable target, cyclin D1, traditionally considered undruggable will be used. However, within the cell nucleus, cyclin D1 forms complexes with CDK6 and CDK4. By conducting a detailed spatial organization-activity relationship study ubiquitination of cyclin D1 while employing the traditional CDK6 inhibitor palbociclib can be achieved.

### Example 10

### Stability Studies of a Representative DNA-PROTAC Under Biological Conditions.

Assessing the stability of branched DNA-PROTACs can be performed using PAGE gel migration and melting point curve analysis. To enhance branched structure stability, flexible junction points can be incorporated into the DNA-PROTAC design. These linkers may comprise alkyl carbon chains or PEG spacers. A self-assembly of DNA-PROTAC can be done in one pot from their constituent individual strands. The flexible linkers at junction points are also expected to improve resistance against nuclease degradation. For in vitro and in vivo application of DNA-PROTAC, it is necessary to stabilize the DNA scaffold against the nucleases and chemical degradations characteristic of physiological conditions. Chemical modifications such as sugar modifications (e.g., locked nucleic acid, threose nucleic acid), and backbone modifications (e.g., phosphorathionate) can be used to stabilize the DNA-PROTACs against nuclease degradation. The integrity of DNA scaffolds consisting of modified DNA strands can be evaluated in physiological salt concentration (1 mM Mg²⁺, 100 mM Na⁺) and against different concentrations of DNA nucleases. The stability kinetics is evaluated using time-dependent reverse phase HPLC and ESI-MS analysis.

Given that ligands for many “undruggable” proteins are lacking, targeting “connector proteins” can be a promising strategy. Potential pitfalls and alternative strategies are as follows: 1) One potential risk with DNA-PROTAC is binding of unintended proteins with the DNA backbone, either in a sequence specific manner or through local DNA structures. Such unintended binders might get degraded once they bind to the DNA backbone and get ubiquitinated by virtue of proximity to the E3 ligase. To address this issue, DNA-PROTAC sequences can be designed to ensure that only the targeted proteins are degraded. 2) To understand E3 ligase and POI interaction in more detail, an all-atom simulation and molecular docking simulations with DNA-PROTAC and bound proteins can be performed. 3) Conjugating small molecules with DNA may potentially affect protein binding. In such instances, the length of the chemical linker that connects small molecule inhibitors to the DNA backbone can be modulated to mitigate such a pitfall. By employing surface plasmon resonance (SPR) analysis, the binding constant (kD) of DNA strands functionalized with small molecules in relation to proteins can be analyzed to evaluate the stability of ternary complex formation. 4) The present approach for linking small molecules is primarily constrained to click chemistry. Direct attachment of small molecules to the DNA backbone using phosphoramidite chemistry can be employed using the appropriate corresponding modifier (available from Glen Research, Trilink, etc.). This advancement will facilitate the ability to make multiple modifications to ligands on the same DNA strand, consequently enhancing resolution to less than 1 nm.

### Example 11

### Synthesis of 4-((2-(2-(2-azidoethoxy) ethoxy)ethyl)amino)-2-(2,6-dioxopiperidin-3-yl) isoindoline-1,3-dione (Azido-PEG3-pomalidomide; E3-i)

The Azido-PEG3-pomalidomide molecule was synthesized using previously published protocol with slight modification. 4-Fluorothalidomide (0.100 g, 0.362 mmol, 1 equiv.), Azido-PEG2-amine (0.082 g, 0.471 mmol, 1.3 equiv.), and N,N-Diisopropylethylamine (189 UL, 1.086 mmol, 3 equiv.) were added into anhydrous dimethyl sulfoxide (1 mL) and reaction mixture was heated 100° C. for overnight under argon atmosphere. Thereafter solvent was co-evaporated with ethanol several times. The crude mixture was purified on a flash chromatography, eluting with 1:1 hexane:ethylacetate (50:50) to obtain yellow-orange solid powder as a product: yield (0.095 g, 0.220 mmol, 61.03%); 1H NMR (500 MHZ, CDCl₃) δ 8.51 (s, 1H), 7.49 (dd, J=8.5, 7.1 Hz, 1H), 7.10 (d, J=7.1 Hz, 1H), 6.92 (d, J=8.5 Hz, 1H), 6.50 (t, J=5.6 Hz, 1H), 5.00-4.89 (m, 1H), 3.74 (t, J=5.4 Hz, 2H), 3.69 (d, J=4.1 Hz, 6H), 3.48 (q, J=5.5 Hz, 2H), 3.38 (t, J=5.0 Hz, 2H), 2.91-2.67 (m, 3H), 2.11 (ddd, J=9.7, 7.2, 3.1 Hz, 1H) (FIG. 9); QTOF-LC/MS (ESI+): m/z calculated for C₁₉H₂₂N₆O₆: 430.16. [M+Na]⁺; found 453.15.

### Example 12

### Synthesis of 6-acetyl-2-((5-(4-(2-(2-(2-azidoethoxy) ethoxy)ethyl) piperazin-1-yl)pyridin-2-yl)amino)-8-cyclopentyl-5-methylpyrido[2,3-d]pyrimidin-7 (8H)-one (Azido-PEG3-palbociclib; POI-i)

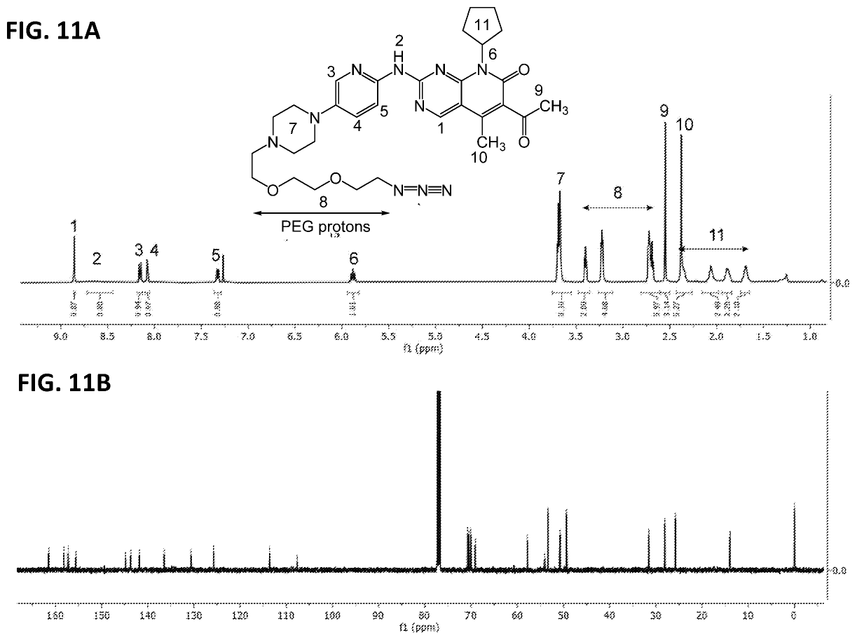

Palbociclib (0.100 g, 0.223 mmol, 1 equiv.), Azide-PEG3-iodide (0.083 g, 0.290 mmol, 1.3 equiv.), Potassium carbonate (0.077 g, 0.558 mmol, 2.5 equiv.), and catalytic amount of Tetraethylammonium bromide (0.015 g, 0.045 mmol, 0.2 equiv.) were added into anhydrous dimethylformamide (2 mL) and reaction mixture was heated at 90° C. for 6 hours under argon atmosphere (FIG. 11). After that reaction mixture was quenched with addition of distilled water (20 mL) and product was extracted with ethyl acetate (20 mL). Next, the organic phase was washed with brine, water and then dried over sodium sulfate for few minutes. Solvent was evaporated in reduced pressure and crude product was purified on a flash chromatography, eluting with 95:5 dichloromethane:methanol. Product was obtained as bright yellow solid: yield (0.07 g, 0.115 mmol, 52%) 1H NMR (500 MHZ, CDCl₃) δ 8.86 (s, 1H), 8.62 (s, 1H), 8.15 (d, J=9.1 Hz, 1H), 8.08 (d, J=3.0 Hz, 1H), 7.33 (dd, J=9.1, 3.0 Hz, 1H), 5.88 (q, J=8.9 Hz, 1H), 3.68 (qd, J=6.6, 3.6 Hz, 7H), 3.40 (t, J=5.1 Hz, 2H), 3.22 (t, J=5.0 Hz, 4H), 2.71 (dt, J=15.8, 5.4 Hz, 5H), 2.55 (s, 3H), 2.38 (s, 5H), 2.17-2.00 (m, 2H), 1.96-1.83 (m, 2H), 1.75-1.58 (m, 2H), 1.26 (s, 1H) (FIG. 11); QTOF-LC/MS (ESI+): m/z calculated for C₃₀H₄₀N₁₀O₄: 604.32. [M+H]⁺; found 605.33.

### Example 13

### Amine Modified Oligonucleotides Synthesis and Characterization.

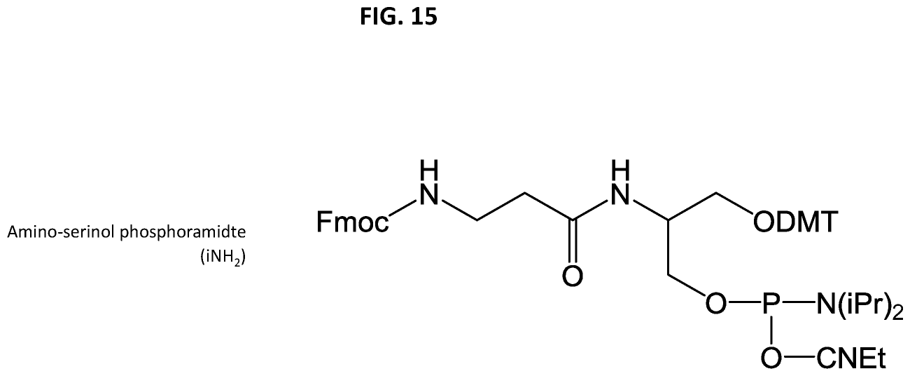

The oligonucleotides were obtained via standard solid-phase oligonucleotide synthesis on a controlled pore glass (CPG, 1 μm). Standard DNA phosphoramidites, solid supports and additional reagents were purchased from Glen Research. The oligonucleotides were synthesized on an Applied Biosystems 3400 automated DNA/RNA synthesizer using a standard 1.0 μmole phosphoramidite cycle of acid-catalyzed detritylation activation and coupling, capping, and iodine oxidation. Stepwise coupling efficiencies and overall yields were determined by automated trityl cation conductivity monitoring. For phosphorothioate modification, 0.05M sulfurizing reagent II is prepared by dissolving in 40 mL pyridine first, followed by 60 mL acetonitrile to form a homogeneous solution. It is connected to the Auxiliary port on the DNA synthesizer and can be used similar to general iodine oxidation. For internal amine modification, amino-serinol phosphoramidite is dissolved in anhydrous acetonitrile to a concentration of 0.1M immediately prior to use. Cleavage of the oligonucleotides from the solid support and deprotection was achieved by exposure to 30% ammonia solution for 120 minutes at 55° C. on a heating-block. The cleavage solutions were diluted with water and ammonia was removed by washing with water using a 100 kDa Amicon filter. The sequences of the oligonucleotides synthesized are listed in Table 2 and Table 3 (where “i-NH₂” denotes the internal amine (depicted in FIG. 15) and * represents phosphorothioate modifications) with the mass spectrometry data.

TABLE 2
DNA strand sequences with internal amine modifiers for subsequent conjugation to
small molecules.

| DNA Strand | | SEQ ID | Calculated | Observed |
|---|---|---|---|---|
| Name | Sequences | NO: | MS | MS |
| Version 01 & 05 | A*-iNH2*- | 8 | 6,309.34 | 6,308.67 |
| (DNA E3-i) | C*T*CCATCTATTCTACG*C*C*A | | | |
| Version 02 to 04 | A*C*T*CCATCTA-iNH2- | 9 | 6,293.34 | 6,292.65 |
| (DNA E3-i) | TTCTACG*C*C*A | | | |
| Version 01 | T*G*G*CGTAGAATAGATGG*A*G*- | 10 | 6,598.34 | 6,597.89 |
| (cDNA POI-i) | iNH2*-T | | | |
| Version 02 | T*G*G*CGTAGAA-iNH2- | 11 | 6,581.53 | 6,582.32 |
| (cDNA POI-i) | TAGATGG*A*G*T | | | |
| Version 03 | T*G*G*CGTAGAATAGA-iNH2- | 12 | 6,581.53 | 6,581.86 |
| (cDNA POI-i) | TGG*A*G*T | | | |
| Version 04 | T*G*G*C-iNH2- | 13 | 6,581.53 | 7,581.72 |
| (cDNA POI-i) | GTAGAATAGATGG*A*G*T | | | |
| Version 05 | T*-iNH2- | 14 | 6,598.34 | 6,597.78 |
| (cDNA POI-i) | G*G*CGTAGAATAGATGG*A*G*T | | | |
| Minor 00 to 03 | A*C*T*CCATCTA-iNH2- | 9 | 6,293.34 | 6,292.65 |
| (DNA E3-i) | TTCTACG*C*C*A | | | |
| Minor 00 (cDNA | T*G*G*CGTAGAATAGA-iNH2- | 12 | 6,581.53 | 6,581.62 |
| POI-i) | TGG*A*G*T | | | |
| Minor 01 (cDNA | T*G*G*CGTAGAATAG-iNH2- | 15 | 6,581.53 | 6,581.62 |
| POI-i) | ATGG*A*G*T | | | |
| Minor 02 (cDNA | T*G*G*CGTAGAATA-iNH2- | 16 | 6,581.53 | 6,581.66 |
| POI-i) | GATGG*A*G*T | | | |
| Minor 03 (cDNA | T*G*G*CGTAGAAT-iNH2- | 17 | 6,581.53 | 6,581.51 |
| POI-i) | AGATGG*A*G*T | | | |
| Version 02 | T*G*G*CGTAGAA-iNH2- | 11 | 6,581.53 | 6,581.61 |
| (CDNA POI-i) | TAGATGG*A*G*T | | | |

TABLE 3
DNA strand sequences with internal amine modifiers for subsequent conjugation to
small molecules and protein of interest binding sites.

| DNA Strand | | SEQ ID | Calculated | Observed |
|---|---|---|---|---|
| Name | Sequences | NO: | MS | MS |
| Version 01 & 05 | A*-E3-i*- | 18 | 7,054.34 | 7,054.66 |
| | C*T*CCATCTATTCTACG*C*C*A | | | |
| Version 02 to 04 | A*C*T*CCATCTA-E3-i- | 3 | 7,037.53 | 7,037.22 |
| | TTCTACG*C*C*A | | | |
| Version 01 | T*G*G*CGTAGAATAGATGG*A*G*- | 19 | 7,518.34 | 7,517.44 |
| | POI-i*-T | | | |
| Version 02 | T*G*G*CGTAGAA-POI-i- | 4 | 7,501.53 | 7,502.34 |
| | TAGATGG*A*G*T | | | |
| Version 03 | T*G*G*CGTAGAATAGA-POI-i- | 5 | 7,501.53 | 7,502.34 |
| | TGG*A*G*T | | | |
| Version 04 | T*G*G*C-POI-i- | 6 | 7,501.53 | 7,501.96 |
| | GTAGAATAGATGG*A*G*T | | | |
| Version 05 | T*-POI-i*- | 20 | 7,518.34 | 7,516.78 |
| | G*G*CGTAGAATAGATGG*A*G*T | | | |
| Minor 00 to 03 | A*C*T*CCATCTA-E3-i- | 3 | 7,037.53 | 7,038.54 |
| | TTCTACG*C*C*A | | | |
| Minor 00 | T*G*G*CGTAGAATAGA-POI-i- | 5 | 7,501.53 | 7,501.85 |
| | TGG*A*G*T | | | |
| Minor 01 | T*G*G*CGTAGAATAG-POI-i- | 21 | 7,501.53 | 7,502.12 |
| | ATGG*A*G*T | | | |
| Minor 02 | T*G*G*CGTAGAATA-POI-i- | 22 | 7,501.53 | 7,501.84 |
| | GATGG*A*G*T | | | |
| Minor 03 | T*G*G*CGTAGAAT-POI-i- | 23 | 7,501.53 | 7,501.96 |
| | AGATGG*A*G*T | | | |
| Version 02 | T*G*G*CGTAGAA-POI-i- | 4 | 7,501.53 | 7,501.85 |
| | TAGATGG*A*G*T | | | |

### Example 14

### Dibenzocyclooctyne (DBCO) Modified Oligonucleotides Synthesis and Characterization.

To conjugate DBCO to an oligonucleotide, an amine-modified DNA strand (Please see DNA sequences used) was treated with 6-fold excess of 200 mM DBCO-sulfo-NHS-ester (dibenzocyclooctyne-sulfo-N-hydroxysuccinimidyl ester) in a 1×PBS buffer at pH ~8.5. The mixture was gently shaken for 3 h at 37° C. The DNA-DBCO conjugates were purified by 3 kDa amicon filter at 8000 rcf by repeated washing (5×) with distilled water to remove the excess small molecules and salts. Following filtration, the mixtures were purified using RP-HPLC and the conjugate peaks verified using ESI-MS.

Synthesis of E3-i and POI-i-DNA conjugates. Conjugates were synthesized using strain promoted alkyne azide cycloaddition (SPAAC) chemistry. Briefly, to DNA-DBCO conjugates (in 1×TAE-12.5 mM MgCl₂, PH 7.5) was added 2 molar equivalents of 10 mM Azido-PEG3-pomalidomide (E3-i), or Azido-PEG3-palbociclib (POI-i) as a solution in DMSO. The reaction mixture was agitated and maintained at 37° C. overnight. The DNA-drug conjugates were purified by 3 kDa amicon filter at 8000 rcf by repeated washing (5×) with distilled water to remove the excess small molecules and salts. Following reaction, the mixture was purified using RP-HPLC and the conjugate peaks verified using ESI-MS.

Purification by RP-HPLC. Following reaction, small molecule-DNA conjugates were purified using a C-18 column on an Agilent 1220 Infinity LC HPLC. The mixtures were purified using a linear gradient method, with Buffer-A (50 mM triethylammonium acetate (TEAA)) and Buffer-B (methanol). A linear gradient was run from 10% to 100% Buffer B over 60 minutes. Conjugates were monitored and collected based on 260 nm (for DNA) and 309 nm (for DBCO) absorbances. Collected fractions were lyophilized overnight until dry.

Intact mass analysis of DTAC conjugates. Collected peaks were tested for purity and identified by quadrupole time-of-flight liquid chromatography mass spectrometry (QTOF LC/MS) analysis in the negative mode. A 50 UM solution is prepared from the stock of small molecule conjugated DNA strands for the analysis using 0.1% ammonium hydroxide as the mobile phase. Identified peaks are deconvoluted in the expected mass range to get the intact mass of the conjugates.

PAGE analysis of DTAC conjugates. Small molecule-DNA conjugates were analyzed using 8% native polyacrylamide gel electrophoresis (PAGE). To each lane was added 15 μL of a 2 μM sample (for single strand oligonucleotides 15 μL of a 10 UM sample was added), and the gel was electrophoresed at 200 V (constant voltage) for 1.5 h at 4° C., then stained with ethidium bromide (EtBr) and imaged with a Bio-Rad Molecular Imager GelDOC XR+ imaging system.

The examples provided herein clearly demonstrate that the compositions of this disclosure can be used to significantly reduce the tumor burden in a subject, resulting in an increased survival time, including up to the end of the experimental study.

Although the foregoing specification and examples fully disclose and enable the embodiments of the present disclosure, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A programmable DNA proteolysis target chimera complex comprising:
a. a first DNA strand comprising one or a plurality of independent E3 ligase ligands; and
b. a second DNA strand comprising one or a plurality of independent protein of interest (POI) targeting ligands,
wherein at least a portion of the first DNA strand is complementary to a portion of the second DNA strand and the first and second DNA strands form a DNA duplex,
wherein the one or a plurality of independent E3 ligase ligands is connected to the first DNA strand at one or a plurality of independently selected positions on said first DNA strand, and
wherein the one or a plurality of independent POI targeting ligands is connected to the second DNA strand at one or a plurality of independently selected positions on said second DNA strand.

2. The complex of claim 1, further comprising a targeting moiety selected from a cell-penetrating peptide or a blood-brain barrier traversal agent.

3. The complex of claim 2, wherein the blood-brain barrier traversal agent is a lipid or cholesterol derivative.

4. The complex of claim 1, wherein the plurality of independent POI targeting ligands target different proteins.

5. The complex of claim 1, wherein the plurality of independent POI targeting ligands target different portions of the same protein.

6. The complex of claim 1, wherein there are at least two independent POI targeting ligands.

7. The complex of claim 1, wherein there are at least two independent E3 ligase ligands.

8. The complex of claim 1, wherein the E3 ligase ligand is covalently connected to the first DNA strand.

9. The complex of claim 1, wherein the E3 ligase ligand is complexed to one or a plurality of E3 ligase proteins.

10. The complex of claim 1, wherein the POI targeting ligand is covalently connected to the second DNA strand.

11. The complex of claim of claim 11, further comprising a protein of interest complexed to the one or plurality of POI targeting ligands.

12. The complex of claim 1, wherein the selected position on said first DNA strand and the selected position on the second DNA strand are separated by a distance from about 0.99 nm to about 7 nm.

13. The complex of claim 1, wherein the selected position on said first DNA strand and the selected position on the second DNA strand are separated by a rotational angle about the double-stranded DNA complex from about 36 to about 180 degrees.

14. The complex of claim 1, wherein the selected position on said first DNA strand and the selected position on the second DNA strand are separated by a distance of about a minor groove to about a major groove.

15. The complex of claim 1, wherein the first DNA strand and second DNA strand independently comprise a nuclease resistance feature.

16. The complex of claim 15, wherein the nuclease resistance feature is selected from a sugar modification or an internucleoside linkage modification.

17. The complex of claim 16, wherein the sugar modification is selected from a locked nucleic acid, a threose nucleic acid, or a 2′-alkoxy modification.

18. The complex of claim 17, wherein the internucleoside linkage modification is a phosphorothioate, phosphoroselenoate, or phosphoramidate.

19. The complex of claim 1, wherein the protein of interest is selected from: CDK6, CDK4, BCR-Abl, EGFR, BTK, BRD4, HDAC6, STAT3, BCL-XI, FAK, P38-alpha, myc, Arora, Ras, and Jak.

20. A method of killing a cancer cell, the method comprising contacting a cancer cell with the complex of claim 1.

21-29. (canceled)


SEQUENCE LISTING (23 sequences)

SEQ ID NO: 1 - DNA, 21 bp, synthetic construct
  source 1..21: mol_type = other DNA
  modified_base 1: mod_base = OTHER; note = Nucleotide with phosphorothioate linkage
  misc_difference 2: note = E3 ligase attached to 3-Dimethoxytrityloxy-2-(3-(fl uorenylmethoxycarbonylamino)propanamido)propyl-1-O-(2-cyan oethyl)-(N,N-diisopropyl)-phosphoramidite modified adenosine, cytidine, thymidine, guanosine, unknown or other nucleotide
  modified_base 3: mod_base = OTHER; note = Nucleotide with phosphorothioate linkage
  modified_base 4: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
anctccatctattctacgcca

SEQ ID NO: 2 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  misc_difference 20: note = Protein of interest attached to 3-Dimethoxytritylox y-2-(3-(fluorenylmethoxycarbonylamino)propanamido)propyl-1 -O-(2-cyanoethyl)-(N,N-diisopropyl)-phosphoramidite modified adenosine, cytidine, thymidine, guanosine, unknown or other nucleotide
tggcgtagaatagatggagnt

SEQ ID NO: 3 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 11: note = E3 ligase attached to 3-Dimethoxytrityloxy-2-(3-(fl uorenylmethoxycarbonylamino)propanamido)propyl-1-O-(2-cyan oethyl)-(N,N-diisopropyl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
actccatctanttctacgcca

SEQ ID NO: 4 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 11: note = Protein of interest attached to 3-Dimethoxytritylox y-2-(3-(fluorenylmethoxycarbonylamino)propanamido)propyl-1 -O-(2-cyanoethyl)-(N,N-diisopropyl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tggcgtagaantagatggagt

SEQ ID NO: 5 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 15: note = Protein of interest attached to 3-Dimethoxytritylox y-2-(3-(fluorenylmethoxycarbonylamino)propanamido)propyl-1 -O-(2-cyanoethyl)-(N,N-diisopropyl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tggcgtagaatagantggagt

SEQ ID NO: 6 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 5: note = Protein of interest attached to 3-Dimethoxytritylox y-2-(3-(fluorenylmethoxycarbonylamino)propanamido)propyl-1 -O-(2-cyanoethyl)-(N,N-diisopropyl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tggcngtagaatagatggagt

SEQ ID NO: 7 - DNA, 21 bp, synthetic construct
  source 1..21: mol_type = other DNA
  modified_base 1: mod_base = OTHER; note = Nucleotide with phosphorothioate linkage
  misc_difference 2: note = Protein of interest attached to 3-Dimethoxytritylox y-2-(3-(fluorenylmethoxycarbonylamino)propanamido)propyl-1 -O-(2-cyanoethyl)-(N,N-diisopropyl)-phosphoramidite modified adenosine, cytidine, thymidine, guanosine, unknown or other nucleotide
  modified_base 3: mod_base = OTHER; note = Nucleotide with phosphorothioate linkage
  modified_base 4: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tnggcgtagaatagatggagt

SEQ ID NO: 8 - DNA, 21 bp, synthetic construct
  source 1..21: mol_type = other DNA
  modified_base 1: mod_base = OTHER; note = Nucleotide with phosphorothioate linkage
  misc_difference 2: note = 3-Dimethoxytrityloxy-2-(3-(fluorenylmethoxycarbonyl amino)propanamido)propyl-1-O-(2-cyanoethyl)-(N,N-diisoprop yl)-phosphoramidite modified adenosine, cytidine, thymidine, guanosine, unknown or other nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
anctccatctattctacgcca

SEQ ID NO: 9 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 11: note = 3-Dimethoxytrityloxy-2-(3-(fluorenylmethoxycarbonyl amino)propanamido)propyl-1-O-(2-cyanoethyl)-(N,N-diisoprop yl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
actccatctanttctacgcca

SEQ ID NO: 10 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  misc_difference 20: note = 3-Dimethoxytrityloxy-2-(3-(fluorenylmethoxycarbonyl amino)propanamido)propyl-1-O-(2-cyanoethyl)-(N,N-diisoprop yl)-phosphoramidite modified adenosine, cytidine, thymidine, guanosine, unknown or other nucleotide with phosphorothioate linkage
tggcgtagaatagatggagnt

SEQ ID NO: 11 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 11: note = 3-Dimethoxytrityloxy-2-(3-(fluorenylmethoxycarbonyl amino)propanamido)propyl-1-O-(2-cyanoethyl)-(N,N-diisoprop yl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tggcgtagaantagatggagt

SEQ ID NO: 12 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 15: note = 3-Dimethoxytrityloxy-2-(3-(fluorenylmethoxycarbonyl amino)propanamido)propyl-1-O-(2-cyanoethyl)-(N,N-diisoprop yl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tggcgtagaatagantggagt

SEQ ID NO: 13 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 5: note = 3-Dimethoxytrityloxy-2-(3-(fluorenylmethoxycarbonyl amino)propanamido)propyl-1-O-(2-cyanoethyl)-(N,N-diisoprop yl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tggcngtagaatagatggagt

SEQ ID NO: 14 - DNA, 21 bp, synthetic construct
  source 1..21: mol_type = other DNA
  modified_base 1: mod_base = OTHER; note = Nucleotide with phosphorothioate linkage
  misc_difference 2: note = 3-Dimethoxytrityloxy-2-(3-(fluorenylmethoxycarbonyl amino)propanamido)propyl-1-O-(2-cyanoethyl)-(N,N-diisoprop yl)-phosphoramidite modified adenosine, cytidine, thymidine, guanosine, unknown or other nucleotide
  modified_base 3: mod_base = OTHER; note = Nucleotide with phosphorothioate linkage
  modified_base 4: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tnggcgtagaatagatggagt

SEQ ID NO: 15 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 14: note = 3-Dimethoxytrityloxy-2-(3-(fluorenylmethoxycarbonyl amino)propanamido)propyl-1-O-(2-cyanoethyl)-(N,N-diisoprop yl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tggcgtagaatagnatggagt

SEQ ID NO: 16 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 13: note = 3-Dimethoxytrityloxy-2-(3-(fluorenylmethoxycarbonyl amino)propanamido)propyl-1-O-(2-cyanoethyl)-(N,N-diisoprop yl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tggcgtagaatangatggagt

SEQ ID NO: 17 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 12: note = 3-Dimethoxytrityloxy-2-(3-(fluorenylmethoxycarbonyl amino)propanamido)propyl-1-O-(2-cyanoethyl)-(N,N-diisoprop yl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tggcgtagaatnagatggagt

SEQ ID NO: 18 - DNA, 21 bp, synthetic construct
  source 1..21: mol_type = other DNA
  modified_base 1: mod_base = OTHER; note = Nucleotide with phosphorothioate linkage
  misc_difference 2: note = E3 ligase attached to 3-Dimethoxytrityloxy-2-(3-(fl uorenylmethoxycarbonylamino)propanamido)propyl-1-O-(2-cyan oethyl)-(N,N-diisopropyl)-phosphoramidite modified adenosine, cytidine, thymidine, guanosine, unknown or other nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
anctccatctattctacgcca

SEQ ID NO: 19 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  misc_difference 20: note = Protein of interest attached to 3-Dimethoxytritylox y-2-(3-(fluorenylmethoxycarbonylamino)propanamido)propyl-1 -O-(2-cyanoethyl)-(N,N-diisopropyl)-phosphoramidite modified adenosine, cytidine, thymidine, guanosine, unknown or other nucleotide with phosphorothioate linkage
tggcgtagaatagatggagnt

SEQ ID NO: 20 - DNA, 21 bp, synthetic construct
  source 1..21: mol_type = other DNA
  modified_base 1: mod_base = OTHER; note = Nucleotide with phosphorothioate linkage
  misc_difference 2: note = Protein of interest attached to 3-Dimethoxytritylox y-2-(3-(fluorenylmethoxycarbonylamino)propanamido)propyl-1 -O-(2-cyanoethyl)-(N,N-diisopropyl)-phosphoramidite modified adenosine, cytidine, thymidine, guanosine, unknown or other nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tnggcgtagaatagatggagt

SEQ ID NO: 21 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 14: note = Protein of interest attached to 3-Dimethoxytritylox y-2-(3-(fluorenylmethoxycarbonylamino)propanamido)propyl-1 -O-(2-cyanoethyl)-(N,N-diisopropyl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tggcgtagaatagnatggagt

SEQ ID NO: 22 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 13: note = Protein of interest attached to 3-Dimethoxytritylox y-2-(3-(fluorenylmethoxycarbonylamino)propanamido)propyl-1 -O-(2-cyanoethyl)-(N,N-diisopropyl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tggcgtagaatangatggagt

SEQ ID NO: 23 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 12: note = Protein of interest attached to 3-Dimethoxytritylox y-2-(3-(fluorenylmethoxycarbonylamino)propanamido)propyl-1 -O-(2-cyanoethyl)-(N,N-diisopropyl)-phosphoramidite modified adenosine, cytidine, thymidine, guanosine, unknown or other nucleotide
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tggcgtagaatnagatggagt